(19) United States
(12) Patent Application Publication
(10) Pub. No.: US 2025/0101508 A1
(43) Pub. Date: Mar. 27, 2025

(54) COMPOSITIONS AND METHODS RELATED TO TET-ASSISTED PYRIDINE BORANE SEQUENCING FOR CELL-FREE DNA

(71) Applicant: The Chancellor, Masters and Scholard of the University of Oxford, Oxforg (GB)

(72) Inventors: Chunxiao Song, Oxford (GB); Paullna Siejka-Zienska, Oxford (GB); Jingfei Cheng, Oxford (GB); Felix Jackson, Oxford (GB)

(21) Appl. No.: 18/291,109

(22) PCT Filed: Jul. 26, 2022
(86) PCT No.: PCT/IB2022/000420

§ 371 Date: Jan. 22, 2024

Related U.S. Application Data
(60) Provisional application No. 63/203,565, filed on Jul. 27, 2021.

(51) Int. Cl. C12Q 1/6874; C12N 15/10; C12Q 1/6806; C12Q 1/6855; C12Q 1/6886

(52) U.S. Cl. CPC C12Q 1/6874; C12N 15/1065; C12Q 1/6806; C12Q 1/6855; C12Q 1/6886; C12Q 2600/112; C12Q 2600/154; C12Q 2600/156

(57) ABSTRACT

The present disclosure provides compositions and methods related to TET-assisted Pyridine Borane Sequencing (TAPS). In particular, the present disclosure provides optimized TAPS for cfDNA (cfTAPS), which provides high-quality and high-depth whole-genome cell-free methylomes. The compositions and methods provided herein facilitate the acquisition of multimodal information about cfDNA characteristics, including DNA methylation, tissue of origin, and DNA fragmentation for the diagnosis and treatment of disease.

### CROSS-REFERENCE TO RELATED APPLICATIONS

This application claims the benefit of U.S. Provisional Application No. 63/203,565 filed Jul. 27, 2021, the contents of which is incorporated herein by reference in its entirety.

The contents of the electronic sequence listing (sequencelisting.xml; Size: 8,000 bytes; and Date of Creation: Jul. 26, 2022) is herein incorporated by reference in its entirety.

### FIELD

The present disclosure provides compositions and methods related to TET-assisted Pyridine Borane Sequencing (TAPS). In particular, the present disclosure provides optimized TAPS for cfDNA (cfTAPS), which provides high-quality and high-depth whole-genome cell-free methylomes. The compositions and methods provided herein facilitate the acquisition of multimodal information about cfDNA characteristics, including DNA methylation, tissue of origin, and DNA fragmentation for the diagnosis and treatment of disease.

### BACKGROUND

Although recent advances in cancer research offer new ways to treat cancer, early detection still represents the best opportunity for curing cancer. Early-stage treatment not only greatly improves patient survival but also costs considerably less. Circulating cell-free DNA (cfDNA)—the free-floating DNA in blood plasma originating from cell death in various healthy and diseased tissues—holds tremendous potential to develop an early cancer detection assay. Genetic information in cfDNA, such as mutations and copy-number variations (CNVs), demonstrate potential utility for monitoring cancer progression and treatment. However, genetic alterations are challenging to detect given the low fraction of tumor DNA in early-stage disease. Furthermore, genetic alterations are weakly informative about the tissue-of-origin, which is needed to determine the location of malignancy.

In contrast, widespread epigenetic changes such as DNA methylation of both cancer cells and tumor microenvironment occur early in tumorigenesis. Recent studies have shown cfDNA methylation to be one of the most promising biomarkers for early cancer detection, by providing thousands of methylation changes that can be combined to overcome detection limits, and tissue-of-origin information that allows cancer localisation with high confidence. DNA methylation is best determined by a whole-genome, base-resolution, and quantitative sequencing method, such as bisulfite sequencing. However, bisulfite sequencing is DNA damaging and expensive; therefore, current cfDNA methylation sequencing is limited by being low-depth, targeted, or low-resolution and qualitative enrichment-based sequencing, thus imperfectly capturing the cfDNA methylome.

### SUMMARY

Embodiments of the present disclosure include a method of obtaining a methylation signature. In accordance with these embodiments, the method includes isolating cell free DNA (cfDNA) from a sample; preparing a sequencing library comprising the cfDNA; and performing TET-assisted Pyridine Borane Sequencing (TAPS) on the sequencing library to obtain a methylation signature of the cfDNA. In some embodiments, the methylation signature is a whole-genome methylation signature.

In some embodiments, the unique mapping rate resulting from TAPS on the cfDNA is at least 80% and/or the unique deduplicated mapping rate is at least 70%.

In some embodiments, preparing the sequencing library comprises ligating sequencing adapters to the isolated cfDNA.

In some embodiments, carrier DNA is added to the sequencing library prior to performing TAPS.

In some embodiments, the method further comprises identifying at least one methylation biomarker from the cfDNA whole-genome methylation signature, and determining whether the methylation biomarker is indicative of cancer.

In some embodiments, the methylation biomarker comprises a differentially methylated region (DMR).

In some embodiments, the method further comprises classifying the sample based on the DMR as compared to a reference DMR.

In some embodiments, the reference DMR corresponds to a non-cancerous control, or a cancerous control.

In some embodiments, the method further comprises identifying at least one methylation biomarker from the cfDNA whole-genome methylation signature, and determining a tissue-of-origin corresponding to the methylation biomarker.

In some embodiments, the method further comprises classifying the sample based on the tissue-of-origin biomarker.

In some embodiments, the method further comprises identifying a DNA fragmentation profile, and determining whether the fragmentation profile is indicative of cancer.

In some embodiments, the method further comprises identifying at least one sequence variant from the cfDNA, and determining whether the sequence variant is indicative of cancer.

In some embodiments, performing TAPS on the sequencing library to obtain the whole-genome methylation signature comprises identifying 5mC modifications in the cfDNA and providing a quantitative measure for frequency of the 5mC modifications.

In some embodiments, performing TAPS on the sequencing library to obtain the whole-genome methylation signature comprises identifying 5hmC modifications in the cfDNA and providing a quantitative measure for frequency of the 5hmC modifications.

In some embodiments, performing TAPS on the sequencing library to obtain the whole-genome methylation signature comprises identifying 5caC modifications in the cfDNA and providing a quantitative measure for frequency of the 5caC modifications.

In some embodiments, performing TAPS on the sequencing library to obtain the whole-genome methylation signature comprises identifying 5fC modifications in the cfDNA and providing a quantitative measure for frequency of the 5fC modifications.

Embodiments of the present disclosure also include a method of determining whether a subject has cancer using any of the methods described herein. In some embodiments, the cancer comprises hepatocellular carcinoma (HCC) or pancreatic ductal adenocarcinoma (PDAC).

Embodiments of the present disclosure also include a method of determining whether a subject has early stage cancer using any of the methods described herein. In some embodiments, the cancer comprises early stage hepatocellular carcinoma (HCC) or early stage pancreatic ductal adenocarcinoma (PDAC).

In still other preferred embodiments, the present invention provides multimodal methods of analyzing cfDNA in a patient sample comprising: isolating cfDNA from a patient sample; converting 5mC and/or 5hmC residues in the sample to DHU residues to provide a modified cfDNA sample; sequencing the modified cfDNA sample to identify methylated regions in the sample, wherein a cytosine (C) to thymine (T) transition or a cytosine (C) to DHU transition in the modified cfDNA sample as compared to an unmodified reference cfDNA provides the location of either a 5mC or 5hmC in the cfDNA; and performing one or more additional analytical steps on the modified cfDNA selected from the group consisting of: a) determining copy number variation of one or more targets in the modified cfDNA sample; b) determining the tissue of origin or one or more targets in the modified cfDNA sample; c) determining the fragmentation profile of the modified cfDNA sample; and d) identifying one or more single nucleotide mutations in the modified cfDNA sample.

In some embodiments, the step of sequencing the modified cfDNA sample to identify methylated regions in the sample comprising identifying at least one differentially methylated region (DMR).

In some embodiments, the multimodal method further comprises classifying the sample based on the DMR as compared to a reference DMR.

In some embodiments, the reference DMR corresponds to a non-cancerous control, or a cancerous control.

In some embodiments, the step of determining copy number variation (CNV) of one or more targets in the modified cfDNA sample comprises determining the observed read count for a target sequence across the genome by dividing the reference genome into bins and counting the number of reads in each bin.

In some embodiments, the presence of copy number aberrations of greater than 500 kb is indicative of CNV in a patient.

In some embodiments, the step of determining the tissue of origin or one or more targets in the modified cfDNA sample comprises tissue deconvolution of data obtained from sequencing the modified cfDNA sample.

In some embodiments, the tissue deconvolution comprises comparing DNA methylation value identified in the modified cfDNA sample with reference DMRs from two or more different tissues.

In some embodiments, the step of determining the fragmentation profile of the modified cfDNA sample comprises classifying the fragment length and periodicity of fragments in the modified cfDNA sample.

In some embodiments, classifying the length and periodicity of fragments in the modified cfDNA sample further comprises calculating the proportion of cfDNA fragments of from 300 to 500 bp in 10 bp length range bins.

In some embodiments, the step of identifying one or more single nucleotide mutations in the modified cfDNA sample further comprises distinguishing C to T SNPs from 5mC or 5hmC at a specific position in the cfDNA by comparing sequencing results after TAPS, wherein the presence of a T read at the specific position in a compliment to the original bottom strand of the cfDNA is indicative of a C to T SNP and the presence of a C read at the specific position in a compliment to the original bottom strand of the cfDNA is indicative of 5mC or 5hmC.

In some embodiments, two or more of steps a, b, c and d are performed on the modified cfDNA.

In some embodiments, three or more of steps a, b, c and d are performed on the modified cfDNA.

In some embodiments, all of steps a, b, c and d are performed on the modified cfDNA.

In some embodiments, the unique mapping rate resulting from the sequencing step is at least 80% and/or the unique deduplicated mapping rate is at least 70%.

In some embodiments, the sequencing step further comprises preparing a sequencing library comprising the cfDNA by ligating sequencing adapters to the isolated cfDNA.

In some embodiments, carrier DNA is added to the cfDNA.

In some embodiments, the multimodal method provides a cfDNA whole-genome methylation signature and the method further comprises identifying at least one methylation biomarker from the cfDNA whole-genome methylation signature, and determining whether the methylation biomarker is indicative of cancer.

In some embodiments, the multimodal method further comprises identifying 5mC modifications in the cfDNA and providing a quantitative measure for frequency of the 5mC modifications.

In some embodiments, the multimodal method further comprises identifying 5hmC modifications in the cfDNA and providing a quantitative measure for frequency of the 5hmC modifications.

In some embodiments, the multimodal method further comprises identifying 5caC modifications in the cfDNA and providing a quantitative measure for frequency of the 5caC modifications.

In some embodiments, the multimodal method further comprises 5fC modifications in the cfDNA and providing a quantitative measure for frequency of the 5fC modifications.

In some embodiments, the step of converting 5mC and/or 5hmC residues in the sample to DHU residues to provide a modified cfDNA sample comprises oxidizing 5mC and/or 5hmC residues to provide 5caC and/or 5fC residues and reducing the 5caC and/or 5fC residues to DHU residues.

In some embodiments, the step of oxidizing 5mC and/or 5hmC residues to provide 5caC and/or 5fC residues comprises treatment of the sample with a Tet enzyme.

In some embodiments, the step of oxidizing 5mC and/or 5hmC residues to provide 5caC and/or 5fC residues comprises treatment of the sample with a chemical oxidizing agent so that one or more 5fC residues are generated.

In some embodiments, the step of reducing the 5caC and/or 5fC residues to DHU residues comprises treatment of the sample with a borane reducing agent.

Embodiments of the present disclosure also include a method of determining whether a subject has early stage cancer using any of the multimodal methods described herein.

### BRIEF DESCRIPTION OF THE DRAWINGS

FIGS. 1A-1C: cfDNA analysis by TAPS. (A) Schematic representation of the TAPS approach for cfDNA analysis. CfDNA is isolated from 1-3 mL of plasma. 10 ng of cfDNA is ligated to Illumina sequencing adapters and topped up with 100 ng of carrier DNA. Subsequently, 5mC and 5hmC in DNA are oxidized by mTet1CD enzyme to 5caC, reduced by PyBr to DHU and amplified and detected as T in the final sequencing. Computational analysis of TAPS data allows for simultaneous characterization of multiple cfDNA features including DNA methylation, tissue of origin, fragmentation patterns and CNVs. (B) Number of total reads, uniquely mapped reads and uniquely mapped, PCR deduplicated reads in 87 cfDNA TAPS libraries. Total number of reads and mean percentage of uniquely mapped reads and deduplicated reads compared to total reads are shown above the bars. Error bars represent standard error. (C) 5mC conversion rate and false positive rate in 85 cfDNA TAPS libraries based on spike-in controls with modified or unmodified cytosines at the known positions. Each dot represents an individual sample.

FIGS. 2A-2I: cfDNA methylation in clinical samples. (A) Cancer stage distribution of 21 HCC patients and 23 PDAC patients included in the study. (B) Mean per CpG genome modification level in non-cancer controls, HCC and PDAC cfDNA. Each dot represents an individual sample. (C) PCA plot of cfDNA methylation in 1 kb genomic windows in non-cancer controls and HCC. (D) PCA plot of cfDNA methylation in 1 kb genomic windows in non-cancer controls and PDAC. (E) The overrepresentation analysis on the regions correlated most with PC2 for HCC and PC1 for PDAC in regulatory regions. (F) Receiver operating characteristic (ROC) curve of model classification performance based on differentially methylated enhancers in HCC and non-cancer controls (n=51, HCC=21, non-cancer controls=30). (G) LOO cancer prediction scores for HCC and non-cancer controls. Dashed line represents probability score threshold. Samples with a probability score above this threshold were predicted as HCC. (H) ROC curve of model classification performance based on differentially methylated promoters between PDAC and non-cancer controls (n=53, PDAC=23, non-cancer controls=30). (I) LOO cancer prediction scores for PDAC and non-cancer controls. Dashed line represents probability score threshold. Samples with a probability score above this threshold were predicted as PDAC.

FIGS. 3A-3E: cfTAPS enables analysis of tissue of origin and fragmentation patterns in cfDNA. (A) The mean tissue contribution in non-cancer individuals estimated by NNLS. Tissue contributions less than 1.5% are aggregated as ‘Other’. (B) Boxplot showing the estimated liver cancer contribution within non-cancer, HCC and PDAC group. Statistical significance was assessed with a paired t-test. n.s. —not significant. (C) The length distribution of cfDNA fragments in the three groups. For each sample, proportion (P) in 10-base pair intervals of long cfDNA fragments (300-500 bp) was used as fragmentation features for PCA analysis and machine learning. (D) Boxplot showing proportion of short (70-150 bp) and long (300-500 bp) fragments in non-cancer controls, PDAC, and HCC. The Kruskal-Wallis test was performed to test differences in fragment size distribution between groups. Statistically significant differences are marked with an asterisk (*P value<0.05, **P value<0.01, ***P value<0.001, ****P value<0.0001). (E) PCA plot of cfDNA 10 bp-fragment fraction in non-cancer controls and HCC (left panel); and non-cancer controls and PDAC (right panel).

FIGS. 4A-4C: Integrating multimodal features from cfTAPS enhances multi-cancer detection. (A) Heatmap showing individual model performance on multi-cancer prediction and the predicted probabilities for each patient. Each vertical column is a patient. Detection yes/no means patients being correctly classified or misclassified based on a particular feature. Predicted score means the probability of classifying the patients to a specific group based on a particular feature. (B) Schematic detailing the method of integrating multiple features (DNA methylation, tissue contribution and fragmentation fraction) extracted from cfTAPS data for multi-cancer prediction. (C) The actual and predicted patient status calculated in LOO cross-validation.

FIGS. 5A-5D: cfDNA TAPS. (A) Agarose gel of 10 representative cfDNA TAPS libraries after post-amplification clean-up. All cfDNA TAPS libraries were prepared from 10 ng of cfDNA and amplified for 7 PCR cycles. (B) Number of mapped read-pairs for hg38, spike-ins and carrier DNA in 87 cfDNA TAPS libraries. Mean percentage of mapped read-pairs compared to total read-pairs is shown above the bars. Error bars represent standard error. (C) Number of total reads, uniquely mapped reads and uniquely mapped, PCR deduplicated reads in cfDNA WGBS (EGAD00001004317) (24). Total number of reads and mean percentage of uniquely mapped reads and deduplicated reads compared to the total reads are shown above the bars. Error bars represent standard error. (D) Correlation between technical replicates of cfDNA TAPS libraries prepared from the same cfDNA samples sequenced to low depth 2.6×. Methylation was calculated in 100 kb windows.

FIGS. 6A-6I: Global cfDNA methylation patterns in cancer and controls. (A) Age and gender distribution of pancreatitis, cirrhosis, PDAC, HCC and non-cancer control patients included in cfTAPS cohort. (B) Genome-wide distribution of CpG modification in cfDNA in non-cancer controls, HCC and PDAC. Bar plots shows distribution of average CpG modification for each group. Overlaid line plots show CpG methylation distribution in each patient. (C-D) Correlation plots of average cfDNA CpG modification level in HCC patients and (C) tumor size (mm) and (D) tumor stage. (E-F) Correlation plots for PDAC patients and (E) tumor size (mm) and (F) tumor stage. Each dot represents an individual patient. Dashed lines represent the linear trend fitted with linear regression. Shaded area represents 95% confidence intervals of the fitted model. Pearson correlation coefficients (cor) and P values are shown in the plots. (G) Distribution of CpG modification levels over chromosome 4 in cfDNA of non-cancer controls, HCC and PDAC. Each line represents an individual patient. Average CpG modification value was calculated per 1 Mb windows along chromosome 4 and Gaussian-smoothed (smoothing window size 10). (H) Methylation variance in 1 Mb genomic windows in non-cancer controls, HCC and PDAC. (I) PCA plot of cfDNA methylation in 1 kb genomic windows in non-cancer controls and HCC, non-cancer controls and PDAC (Crohn's disease and colitis are coloured in green and yellow respectively).

FIGS. 7A-7I: HCC and PDAC prediction based on cfDNA DMRs. (A) Overview of the LOO model training and validation approach. Total number of samples is labelled as n. At each iteration, the model training set consists of n−1 samples. Differentially methylated enhancers (for HCC) or promoters (for PDAC) were selected for model building. The predictive model was evaluated on the held-out test sample in each fold. Cirrhosis and pancreatitis samples were not included in DMR identification and model building. (B) HCC cancer prediction scores for cirrhosis samples. Each blue dot represents the predicted score for an individual LOO model. The Black dot shows average probability score for a particular sample. The dashed line represents probability score threshold. Samples with average probability score above this threshold were predicted as HCC. (C) Gene Ontology analysis of genes related to differentially methylated enhancers based in HCC cfDNA (P value<0.002) using Enrichr against NCI-Nature Pathway Interaction. Top 10 categories selected based on P value are shown in the graph. Gene-enhancer interactions were assigned using GeneHancer reference database. (D) Methylation of representative differentially methylated enhancer in HCC cfDNA for DLC1 gene (two-tailed t-test P value=8.765e-06). (E) PDAC cancer prediction scores for pancreatitis samples. Each yellow dot represents the predicted score for an individual LOO model. The black dot shows the average probability score for a particular sample. The dashed line represents probability score threshold. Samples with average probability score above this threshold were predicted as PDAC. (F) Gene Ontology analysis of the genes nearest to the differentially methylated promoters in PDAC cfDNA (P value<0.002) using Enrichr against NCI-Nature Pathway Interaction. Top 10 categories selected based on P value are shown on the graph. (G) Methylation of representative differentially methylated promoter in PDAC cfDNA for RB1 gene (two-tailed t-test P value=0.0017). (H) HCC cancer prediction scores for the independent cfDNA WGBS dataset (EGAD00001004317). Each dot represents the predicted score for an individual LOO model. Grey dot belongs to non-cancer controls and the red dot belongs to HCC. The Black dot shows average probability score for a particular sample. The dashed line represents probability score threshold. Samples with average probability score above this threshold were predicted as HCC. (I) Percentage of ref DMRs that can be detected in down-sampled reads. DMRs that were identified in original LOO model training were treated as ref DMRs.

FIGS. 8A-8I: cfDNA tissue of origin. (A) t-SNE plot of reference tissue methylation atlas. (B) The average tissue contribution in HCC and PDAC individuals. (C) Boxplot showing the estimated T cell contribution in non-cancer, HCC and PDAC cfDNA samples. (D) ROC curve of model performance using tissue contribution to classify HCC vs. non-cancer. (E) LOO cancer prediction scores for HCC and non-cancer controls using classifiers trained on tissue contribution. The dashed line represents the probability score threshold. Samples with probability score above this threshold were predicted as HCC. (F) Cancer scores for cirrhosis samples using HCC vs. non-cancer classifiers. Each blue dot represents the predicted scores for an individual model. Black dot shows the average probability score for a particular sample. Dashed line represents probability score threshold. Samples with average probability score above this threshold were predicted as HCC. (G) ROC curve of model performance using tissue contribution to classify PDAC vs control. (H) LOO cancer prediction scores for PDAC and non-cancer controls using classifiers built based on tissue contribution. Dashed line represents probability score threshold. Samples with probability score above this threshold were predicted as PDAC. (I) PDAC Cancer scores for pancreatitis samples using PDAC vs. non-cancer classifiers. Each yellow dot represents the predicted scores for an individual model. Black dot shows the average probability score for a particular sample. Dashed line represents probability score threshold. Samples with average probability score above this threshold were predicted as PDAC.

FIGS. 9A-9B: CNVs analysis in cfDNA. (A) CNV estimation heatmap from cfDNA in 100 kb bin. (B) cfDNA samples with CNV larger than 500 k.

FIGS. 10A-10G: cfDNA fragmentation patterns for cancer prediction. (A) Fragment size distribution of cfDNA in public whole genome bisulfite sequencing data. Frequency was calculated as number of fragments of particular length divided by total number of fragments. (B) ROC curve of HCC and non-cancer control prediction scores from a generalized linear model using proportion of long cfDNA fragments (300-500 bp) in 10 bp bins as features. (C) Cancer prediction scores for HCC and non-cancer controls in classifiers trained using LOO cross-validation. The dashed line represents the probability score threshold. Samples with a probability score above this threshold were predicted as HCC. (D) HCC cancer prediction scores for cirrhosis samples in these classifiers. Each blue dot represents the predicted score for an individual model. Black dots show average prediction score. The dashed line represents probability score threshold: samples with average probability score above this threshold were predicted as HCC. (E) ROC curve of PDAC and non-cancer control prediction scores from a generalized linear model using proportion of long cfDNA fragments (300-500 bp) in 10 bp bins as features. (F) LOO cancer prediction scores for PDAC and non-cancer controls in classifiers built based on cfDNA fragments frequency in 10 bp length range. The dashed line represents the probability score threshold. Samples with probability score above this threshold were predicted as PDAC. (G) PDAC cancer prediction scores for pancreatitis samples in classifiers built based on cfDNA fragments frequency in 10 bp length range. Each yellow dot represents the predicted score for an individual model. Black dots show average prediction score. The dashed line represents probability score threshold: samples with average probability score above this threshold were predicted as PDAC.

FIGS. 11A-11C: Multi-cancer detection with cfTAPS. (A) Methylation, tissue contribution and fragmentation fraction model performance on three-class classification. Upper panel shows the accuracy of each classifier, lower panel shows the actual and predicted patient status in LOO cross-validation analysis. (B) Heatmap showing the methylation status of the selected genomic region used for cancer-type prediction. (C) Gene Ontology analysis using Enrichr against NCI-Nature Pathway Interaction on the nearest genes of the selected DMRs for three class classification.

FIG. 12: Schematic depiction of different patterns derived from C to T SNPs and methylated cytosines in target sequences before and after TAPS. In the diagram OT means Original Top, OB means Original Bottom, CTOT means Complimentary to Original Top, CTOB means Complimentary to Original Bottom.

### DETAILED DESCRIPTION

Recently, TET-assisted Pyridine Borane Sequencing (TAPS), a bisulfite-free DNA methylation sequencing method was developed, as described in International PCT Appln. PCT/US2019/012627, filed Jan. 8, 2019, which claims priority to U.S. Provisional Patent Appln. Nos. 62/614,798, filed Jan. 8, 2018; 62/660,523, filed Apr. 20, 2018; and 62/771,409, filed Nov. 26, 2018, each of which is incorporated herein by reference in its entirety. TAPS is based on the use of mild chemistry to detect DNA methylation directly and demonstrated improved sequence quality, mapping rate and coverage compared to bisulfite sequencing, while reducing sequencing cost by half. The combination of direct methylation detection and the non-destructive nature of TAPS makes it useful not only for DNA methylation analysis, but also for simultaneous genetic analysis in cfDNA, as described further herein, which could enhance non-invasive cancer detection by liquid biopsies. Embodiments of the present disclosure include optimized TAPS for cfDNA (cfTAPS) to deliver high-quality and high-depth whole-genome methylome from as low as 10 ng cfDNA.

As described further herein, cfTAPS was applied to hepatocellular carcinoma (HCC) and pancreatic ductal adenocarcinoma (PDAC) cfDNA, two cancer types with particularly poor prognosis, mostly due to detection at an advanced disease stage. Non-invasive methods for early detection of PDAC and HCC are not available, which contributes to their late diagnosis. For decades, HCC detection has relied on liver ultrasound, combined with serum α-fetoprotein (AFP) measurements. However, these methods have low specificity and sensitivity. There is no blood test to detect or diagnose PDAC. Carbohydrate antigen 19-9 (CA19-9) is used for monitoring PDAC treatment and development, but its sensitivity and specificity are too low to diagnose or screen for PDAC. Therefore, novel approaches for PDAC and HCC detection are urgently needed.

Results provided herein demonstrate that the rich information from cfTAPS enables integrated multimodal epigenetic and genetic analysis of differential methylation, tissue of origin, and fragmentation profiles to accurately distinguish cfDNA samples from patients with HCC and PDAC from controls and patients with pre-cancerous inflammatory conditions. Additionally, results provided herein demonstrate the successful optimization and application of cfTAPS to characterize whole-genome base-resolution methylome in cfDNA from HCC, PDAC and non-cancer controls. Using just 10 ng cfDNA, cfTAPS libraries demonstrated greatly improved sequencing quality and depth compared to previous cfDNA WGBS. Indeed, using less cfDNA input than previous studies, cfDNA TAPS generated the most comprehensive cell-free methylation to date. The much higher yield of informative reads allows cfTAPS to extract more information from a given amount of cfDNA and makes it a viable option for large-scale cfDNA methylation studies. The use of TAPS resulted in superior unique mapping rates and deduplicated unique mapping rates as compared to other methods. In some embodiments, the unique mapping rate is at least 65% and/or the unique deduplicated mapping rate is at least 55%. In some embodiments, the unique mapping rate is at least 70% and/or the unique deduplicated mapping rate is at least 60%. In some embodiments, the unique mapping rate is at least 75% and/or the unique deduplicated mapping rate is at least 65%. In some embodiments, the unique mapping rate is at least 80% and/or the unique deduplicated mapping rate is at least 70%. In some embodiments, the unique mapping rate is at least 85% and/or the unique deduplicated mapping rate is at least 72%. In some embodiments, the unique mapping rate is at least 90% and/or the unique deduplicated mapping rate is at least 75%.

The deep sequencing achieved by cfTAPS enables detailed analysis of the cell-free methylome and whole-genome discovery of methylation biomarkers for early cancer detection. While significant global hypomethylation was not observed, suggesting that the fraction of cfDNA derived from tumor cells is low (as corroborated by the lack of CNVs in most cancer patients included herein), results indicated that local methylation signals in regulatory regions such as enhancers and promoters contained cancer-specific information that could accurately distinguish HCC and PDAC from controls. This is particularly significant considering the inflammation-enriched real-world control group used in the patient cohort and that the HCC model disclosed herein can correctly identify all HCC and control patients from a cfDNA WGBS dataset as an independent validation.

Another important advantage of cfDNA methylation for early cancer detection is the ability to determine tissue-of-origin information. Using currently available public WGBS tissue databases, a whole-genome tissue deconvolution of cfTAPS data was performed, and results indicated increased liver tumor contribution in HCC cfDNA and distinct immune signatures in cancer cfDNA. The tissue deconvolution itself can be used for cancer detection. Finally, since TAPS converts modified cytosine directly, it maximally retains the underlying genetic information compared to other approaches that convert unmodified cytosines. In the present disclosure, CNVs and fragmentation information was extracted from cfTAPS, the latter of which is lost in cfDNA WGBS. Results further demonstrated that an integrated approach combining differential methylation, tissue of origin and fragmentation profiles could improve the model performance for multi-cancer detection.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide.

As used herein, a “methylated nucleic acid molecule” refers to a nucleic acid molecule that contains one or more methylated nucleotides.

As used herein, a “methylation state”, “methylation profile”, “methylation status,” and “methylation signature” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

As used herein, “methylation frequency” or “methylation percent (%)” refer to the number of instances in which a molecule or locus is methylated relative to the number of instances the molecule or locus is unmethylated. Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein, the term “whole-genome cfDNA methylation signature” refers to a signature obtained through any method that looks across the entire breadth of the genome for candidate methylation markers, rather than a narrow few candidate sites (as with an array based technology).

As used herein, the term “copy number variation” (abbreviated CNV) refers to a circumstance in which the number of copies of a specific segment of DNA varies among different individuals' genomes.

As used herein, the term “unique mapping rate” refers to a metric used in validation of sequencing data, and specifically the percentage of sequencing reads that map to exactly one location within the reference genome. In some embodiments, the unique mapping rate may be calculated as the proportion of reads (e.g., with MAPQ>=1 using bwa align) with defined parameters (e.g., 500,120,1000,20) compared to total number of sequenced reads.

As used herein, the term “unique deduplicated mapping rate” refers to the percentage of deduplicated sequencing reads (after removing the duplicates) that map to exactly one location within the reference genome. In some preferred embodiments, the unique deduplicated mapping rate may be determined by calculating the proportion of properly mapped reads after removing PCR duplicates (e.g., with MarkDuplicates (Picard)) compared to total number of sequenced reads.

As used herein, the term “tissue deconvolution” refers to sorting sequenced cfDNA in a sample into its tissues of origin, and determining the relative contribution from the tissues. In some preferred embodiments, cfDNA methylation is compared to methylation values in a reference atlas (e.g., at DMRs). These methods preferably use a regression method where cfDNA origin proportions are regression coefficients.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses.

### 2. TET-ASSISTED PYRIDINE BORANE SEQUENCING (TAPS)

Embodiments of the present disclosure provide a bisulfite-free, base-resolution method for detecting 5-methylcytosine (5mC) and 5-hydroxymethylcytosine (5hmC) in a sequence (TAPS), including for use with circulating cell free DNA. As disclosed in in International PCT Appln. PCT/US2019/012627 (filed Jan. 8, 2019, which claims priority to U.S. Provisional Patent Appln. Nos. 62/614,798, filed Jan. 8, 2018; 62/660,523, filed Apr. 20, 2018; and 62/771,409, filed Nov. 26, 2018, each of which is incorporated herein by reference in its entirety), TAPS comprises the use of mild enzymatic and chemical reactions to detect 5mC and 5hmC directly and quantitatively at base-resolution without affecting unmodified cytosine. The present disclosure also provides methods to detect 5-formylcytosine (5fC) and 5-carboxylcytosine (5caC) at base resolution without affecting unmodified cytosine. Thus, the methods provided herein provide mapping of 5mC, 5hmC, 5fC and 5caC and overcome the disadvantages of previous methods such as bisulfite sequencing.

Methods for Identifying 5mC. In some embodiments, the methods of the present disclosure include identifying 5mC in a DNA sample (targeted DNA or whole-genome), and providing a quantitative measure for the frequency of the 5mC modification at each location where the modification was identified in the DNA. In some embodiments, the percentages of the T at each transition location provide a quantitative level of 5mC at each location in the DNA. In accordance with these embodiments, methods for identifying 5mC can include the use of a blocking group. In other embodiments, methods for identifying 5mC do not require the use of a blocking group (e.g., cfTAPS described further below).

When a blocking group is used to identify 5mC in a DNA (e.g., cfDNA) without including 5hmC, the 5hmC in the sample is blocked so that it is not subject to conversion to 5caC and/or 5fC. In some embodiments, the 5hmC in the sample DNA are rendered non-reactive to the subsequent steps by adding a blocking group to the 5hmC. In one embodiment, the blocking group is a sugar, including a modified sugar, for example glucose or 6-azide-glucose (6-azido-6-deoxy-D-glucose). The sugar blocking group can be added to the hydroxymethyl group of 5hmC by contacting the DNA sample with uridine diphosphate (UDP)-sugar in the presence of one or more glucosyltransferase enzymes. In some embodiments, the glucosyltransferase is T4 bacteriophage β-glucosyltransferase (βGT), T4 bacteriophage α-glucosyltransferase (αGT), and derivatives and analogs thereof βGT is an enzyme that catalyzes a chemical reaction in which a beta-D-glucosyl (glucose) residue is transferred from UDP-glucose to a 5-hydroxymethylcytosine residue in a nucleic acid.

Methodsfor Identifying 5hmC. In some embodiments, the methods of the present disclosure include identifying 5mC or 5hmC in a DNA sample (targeted DNA or whole-genome). In some embodiments, the method provides a quantitative measure for the frequency the of 5mC or 5hmC modifications at each location where the modifications were identified in the DNA. In some embodiments, the percentages of the T at each transition location provide a quantitative level of 5mC or 5hmC at each location in the DNA. In accordance with these embodiments, the method for identifying 5mC or 5hmC provides the location of 5mC and 5hmC, but does not distinguish between the two cytosine modifications. Rather, both 5mC and 5hmC are converted to DHU. The presence of DHU can be detected directly, or the modified DNA can be replicated by known methods where the DHU is converted to T. In some embodiments, methods for identifying 5hmC include the use of a blocking group. In other embodiments, methods for identifying 5hmC do not require the use of a blocking group (e.g., cfTAPS described further below).

Methods for Identifying 5mC and/or Identifying 5hmC. The present disclosure provides a method for identifying 5mC and identifying 5hmC in a DNA (e.g., cfDNA) by performing the method for identifying 5mC on a first DNA sample, and performing the method for identifying 5mC or 5hmC on a second DNA sample. In some embodiments, the first and second DNA samples are derived from the same DNA sample. For example, the first and second samples may be separate aliquots taken from a sample comprising DNA to be analyzed (e.g., cfDNA).

Because the 5mC and 5hmC (that is not blocked) are converted to 5fC and 5caC before conversion to DHU, any existing 5fC and 5caC in the DNA sample will be detected as 5mC and/or 5hmC. However, given the extremely low levels of 5fC and 5caC in genomic DNA under normal conditions, this will often be acceptable when analyzing methylation and hydroxymethylation in a DNA sample. The 5fC and 5caC signals can be eliminated by protecting the 5fC and 5caC from conversion to DHU by, for example, hydroxylamine conjugation and EDC coupling, respectively. In accordance with these embodiments, the method identifies the locations and percentages of 5hmC in the DNA through the comparison of 5mC locations and percentages with the locations and percentages of 5mC or 5hmC (together). Alternatively, the location and frequency of 5hmC modifications in a DNA can be measured directly.

In some embodiments, the step of converting the 5hmC to 5fC comprises oxidizing the 5hmC to 5fC by contacting the DNA with, for example, potassium perruthenate (KRuO₄) (as described in Science. 2012, 33, 934-937 and WO2013017853, incorporated herein by reference); or Cu(II)/TEMPO (copper(II) perchlorate and 2,2,6,6-tetramethylpiperidine-1-oxyl (TEMPO)) (as described in Chem. Commun., 2017, 53, 5756-5759 and WO2017039002, incorporated herein by reference). The 5fC in the DNA sample is then converted to DHU by the methods disclosed herein (e.g., by the borane reaction).

In some embodiments, identifying 5fC and/or 5caC provides the location of 5fC and/or 5caC, but does not distinguish between these two cytosine modifications. Rather, both 5fC and 5caC are converted to DHU, which is detected by the methods described herein.

Methods for Identifying 5caC. In some embodiments, the method includes identifying 5caC in a DNA sample (targeted DNA or whole-genome), and provides a quantitative measure for the frequency the of 5caC modification at each location where the modification was identified in the DNA. In some embodiments, the percentages of the T at each transition location provide a quantitative level of 5caC at each location in the DNA. In accordance with these embodiments, methods for identifying 5caC can include the use of a blocking group. In other embodiments, methods for identifying 5caC do not require the use of a blocking group (e.g., cfTAPS described further below).

In some embodiments, when the 5fC is blocked (and 5mC and 5hmC are not converted to DHU), the identification of 5caC in the DNA can occur. In some embodiments, adding a blocking group to the 5fC in the DNA sample comprises contacting the DNA with an aldehyde reactive compound including, for example, hydroxylamine derivatives, hydrazine derivatives, and hyrazide derivatives. Hydroxylamine derivatives include ashydroxylamine; hydroxylamine hydrochloride; hydroxylammonium acid sulfate; hydroxylamine phosphate; O-methylhydroxylamine; O-hexylhydroxylamine; O-pentylhydroxylamine; O-benzylhydroxylamine; and particularly, O-ethylhydroxylamine (EtONH2), O-alkylated or O-arylated hydroxylamine, acid or salts thereof. Hydrazine derivatives include N-alkylhydrazine, N-arylhydrazine, N-benzylhydrazine, N,N-dialkylhydrazine, N,N-diarylhydrazine, N,N-dibenzylhydrazine, N,N-alkylbenzylhydrazine, N,N-arylbenzylhydrazine, and N,N-alkylarylhydrazine. Hydrazide derivatives include -toluenesulfonylhydrazide, N-acylhydrazide, N,N-alkylacylhydrazide, N,N-benzylacylhydrazide, N,N-arylacylhydrazide, N-sulfonylhydrazide, N,N-alkylsulfonylhydrazide, N,N-benzylsulfonylhydrazide, and N,N-arylsulfonylhydrazide.

Methods for Identifying 5fC. In some embodiments, the method includes identifying 5fC in a DNA sample (targeted DNA or whole-genome), and provides a quantitative measure for the frequency the of 5fC modification at each location where the modification was identified in the DNA. In some embodiments, the percentages of the T at each transition location provide a quantitative level of 5fC at each location in the DNA. In accordance with these embodiments, methods for identifying 5fC can include the use of a blocking group. In other embodiments, methods for identifying 5fC do not require the use of a blocking group (e.g., cfTAPS described further below).

In some embodiments, adding a blocking group to the 5caC in the DNA sample can be accomplished by (i) contacting the DNA sample with a coupling agent, for example a carboxylic acid derivatization reagent like carbodiimide derivatives such as 1-ethyl-3-(3-dimethylaminopropyl)carbodiimide (EDC) or N,N′-dicyclohexylcarbodiimide (DCC), and (ii) contacting the DNA sample with an amine, hydrazine or hydroxylamine compound. Thus, for example, 5caC can be blocked by treating the DNA sample with EDC and then benzylamine, ethylamine, or another amine to form an amide that blocks 5caC from conversion to DHU (e.g., by pic-BH₃).

### 3. TAPS FOR CFDNA (CFTAPS)

The present disclosure provides optimized TAPS for cfDNA (cfTAPS) to provide high-quality and high-depth whole-genome cell-free methylomes. As described further below, in one embodiment of the present disclosure, cfTAPS was applied to 85 cfDNA samples from patients with hepatocellular carcinoma (HCC) or pancreatic ductal adenocarcinoma (PDAC) and non-cancer controls. From just 10 ng of cfDNA (1-3 mL of plasma), the most comprehensive cfDNA methylome to date was generated. The results provided herein demonstrated that cfTAPS provides multimodal information about cfDNA characteristics, including DNA methylation, tissue of origin, and DNA fragmentation. Integrated analysis of these epigenetic and genetic features enables accurate identification of early HCC and PDAC. Because the methods of the present disclosure utilize mild enzymatic and chemical reactions that avoid the substantial degradation of nucleic acids associated with methods like bisulfite sequencing, the methods of the present disclosure are useful in analysis of low-input samples, such as circulating cell-free DNA and in single-cell analysis.

In accordance with these embodiments, the present disclosure provides a method of obtaining a methylation signature. In some embodiments, the method includes isolating cell free DNA (cfDNA) from a sample; preparing a sequencing library comprising the cfDNA; and performing TET-assisted Pyridine Borane Sequencing (TAPS) on the sequencing library to obtain a methylation signature of the cfDNA. In some embodiments, the methylation signature is a whole-genome methylation signature.

In some embodiments, preparing the sequencing library comprises ligating sequencing adapters to the isolated cfDNA to facilitate performing a sequencing reaction. In some embodiments, carrier nucleic acids or a mix of carrier nucleic acids (e.g., DNA) are added to the sequencing library prior to performing TAPS. Carrier nucleic acids can be any specific or non-specific DNA molecules (or nucleic acid derivatives thereof) that enhance one or more aspects of cfDNA recovery from a sample. In some embodiments, carrier DNA comprises a DNA molecule having a specific sequence; and in other embodiments, carrier DNA comprises a mix of DNA molecules having different sequences. In some embodiments, carrier DNA can include DNA with the following sequence, including any fragments and/or derivatives thereof:

(SEQ ID NO: 1)
AGGCAACTTTATGCCCATGCAACAGAAACTATAAAAAATACAGAGAATG
AAAAGAAACAGATAGATTTTTTAGTTCTTTAGGCCCGTAGTCTGCAAAT
CCTTTTATGATTTTCTATCAAACAAAAGAGGAAAATAGACCAGTTGCAA
TCCAAACGAGAGTCTAATAGAATGAGGTCGAAAAGTAAATCGCGCGGGT
TTGTTACTGATAAAGCAGGCAAGACCTAAAATGTGTAAAGGGCAAAGTG
TATACTTTGGCGTCACCCCTTACATATTTTAGGTCTTTTTTTATTGTGC
GTAACTAACTTGCCATCTTCAAACAGGAGGGCTGGAAGAAGCAGACCGC
TAACACAGTACATAAAAAAGGAGACATGAACGATGAACATCAAAAAGTT
TGCAAAACAAGCAACAGTATTAACCTTTACTACCGCACTGCTGGCAGGA
GGCGCAACTCAAGCGTTTGCGAAAGAAACGAACCAAAAGCCATATAAGG
AAACATACGGCATTTCCCATATTACACGCCATGATATGCTGCAAATCCC
TGAACAGCAAAAAAATGAAAAATATAAAGTTCCTGAGTTCGATTCGTCC
ACAATTAAAAATATCTCTTCTGCAAAAGGCCTGGACGTTTGGGACAGCT
GGCCATTACAAAACACTGACGGCACTGTCGCAAACTATCACGGCTACCA
CATCGTCTTTGCATTAGCCGGAGATCCTAAAAATGCGGATGACACATCG
ATTTACATGTTCTATCAAAAAGTCGGCGAAACTTCTATTGACAGCTGGA
AAAACGCTGGCCGCGTCTTTAAAGACAGCGACAAATTCGATGCAAATGA
TTCTATCCTAAAAGACCAAACACAAGAATGGTCAGGTTCAGCCACATTT
ACATCTGACGGAAAAATCCGTTTATTCTACACTGATTTCTCCGGTAAAC
ATTACGGCAAACAAACACTGACAACTGCACAAGTTAACGTATCAGCATC
AGACAGCTCTTTGAACATCAACGGTGTAGAGGATTATAAATCAATCTTT
GACGGTGACGGAAAAACGTATCAAAATGTACAGCAGTTCATCGATGAAG
GCAACTACAGCTCAGGCGACAACCATACGCTGAGAGATCCTCACTACGT
AGAAGATAAAGGCCACAAATACTTAGTATTTGAAGCAAACACTGGAACT
GAAGATGGCTACCAAGGCGAAGAATCTTTATTTAACAAAGCATACTATG
GCAAAAGCACATCATTCTTCCGTCAAGAAAGTCAAAAACTTCTGCAAAG
CGATAAAAAACGCACGGCTGAGTTAGCAAACGGCGCTCTCGGTATGATT
GAGCTAAACGATGATTACACACTGAAAAAAGTGATGAAACCGCTGATTG
CATCTAACACAGTAACAGATGAAATTGAACGCGCGAACGTCTTTAAAAT
GAACGGCAAATGGTACCTGTTCACTGACTCCCGCGGATCAAAAATGACG
ATTGACGGCATTACGTCTAACGATATTTACATGCTTGGTTATGTTTCTA
ATTCTTTAACTGGCCCATACAAGCCGCTGAACAAAACTGGCCTTGTGTT
AAAAATGGATCTTGATCCTAACGATGTAACCTTTACTTACTCACACTTC
GCTGTACCTCAAGCGAAAGGAAACAATGTCGTGATTACAAGCTATATGA
CAAACAGAGGATTCTACGCAGACAAACAATCAACGTTTGCGCCTAGCTT
CCTGCTGAACATCAAAGGCAAGAAAACATCTGTTGTCAAAGACAGCATC
CTTGAACAAGGACAATTAACAGTTAACAAATAAAAACGCAAAAGAAAAT
GCCGATATCCTATTGGCATTGACGGTCTCCAGTAAAGGTGGATACGGAT
CCGAATTCGAGCTCCGTCGACAAGCTTGCGGCCGCACTCGAGCACCACC
ACCACCACCACTGAGATCCGGCTGCTAACAAAGCCCGAAAGGAAGCTGA
GTTGGCTGCTGCCACCGCTGAGCAATAACTAGCATAACCCCTTGG.

In some embodiments, the use of carrier DNA results in higher library yields. As would be recognized by one of ordinary skill in the art based on the present disclosure, carrier DNA can be obtained by any means known in the art, including but not limited to, PCR amplification from a vector or plasmid template using one or more primers. In some embodiments, at least 1 ng of carrier DNA can be used. In some embodiments, at least 10 ng of carrier DNA can be used. In some embodiments, at least 25 ng of carrier DNA can be used. In some embodiments, at least 50 ng of carrier DNA can be used. In some embodiments, at least 100 ng of carrier DNA can be used. In some embodiments, at least 150 ng of carrier DNA can be used. In some embodiments, at least 200 ng of carrier DNA can be used. In some embodiments, at least 250 ng of carrier DNA can be used. In some embodiments, at least 500 ng of carrier DNA can be used. In some embodiments, about 1 ng to about 500 ng of carrier DNA can be used. In some embodiments, about 1 ng to about 500 ng of carrier DNA can be used. In some embodiments, about 50 ng to about 250 ng of carrier DNA can be used. In some embodiments, about 75 ng to about 150 ng of carrier DNA can be used. In some embodiments, about 50 ng to about 150 ng of carrier DNA can be used. In some embodiments, about 75 ng to about 125 ng of carrier DNA can be used.

In some embodiments, and as described herein, the method further comprises identifying at least one methylation biomarker from the cfDNA whole-genome methylation signature, and determining whether the methylation biomarker is indicative of cancer. In some embodiments, the methylation biomarker comprises a differentially methylated region (DMR). In some embodiments, the method further comprises classifying the sample based on the DMR as compared to a reference DMR. In some embodiments, the reference DMR corresponds to a non-cancerous control, or a cancerous control.

In some embodiments, and as described herein, the method further comprises identifying at least one methylation biomarker from the cfDNA whole-genome methylation signature, and determining a tissue-of-origin corresponding to the methylation biomarker. In some embodiments, the method further comprises classifying the sample based on the tissue-of-origin biomarker.

In some embodiments, and as described herein, the method further comprises identifying a DNA fragmentation profile, and determining whether the fragmentation profile is indicative of cancer. In accordance with these embodiments, DNA fragmentation profile can be determined from cfTAPS whole genome sequencing data (e.g., read pair alignment positions). In some preferred embodiments, sequenced reads from cfTAPS are first aligned to a reference genome. The length of cfDNA fragment is then extracted from alignment files produced from the sequencing data. The proportion in 10-bp intervals of cfDNA fragments is used as the fragmentation profile of the cell free DNA.

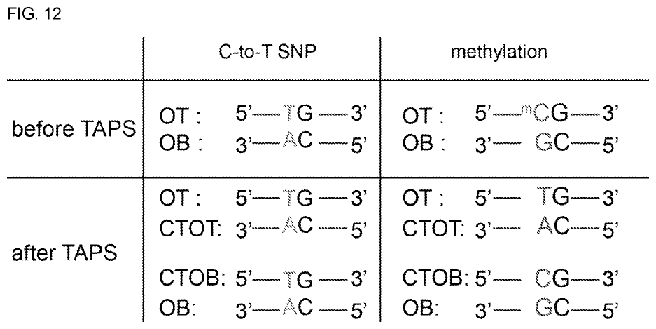

In some embodiments, the method further comprises identifying at least one sequence variant from the cfDNA, and determining whether the sequence variant is indicative of cancer. For example, in some embodiments, cfTAPS can also differentiate methylation from C-to-T genetic variants or single nucleotide polymorphisms (SNPs), and therefore, can be used to detect genetic variants. In some embodiments, methylations and C-to-T SNPs can result in different patterns in cfTAPS. For example, methylations can result in T/G reads in an original top strand/original bottom strand, and A/C reads in strands complementary to these. In some embodiments, C-to-T SNPs can result in T/A reads in an original top strand/original bottom strand and strands complementary to these. These different patterns are illustrated in FIG. 12. This further increases the utility of cfTAPS in providing both methylation information and genetic variants, and therefore mutations, in one experiment and sequencing run. This ability of the cfTAPS methods disclosed herein provides integration of genomic analysis with epigenetic analysis, and a substantial reduction of sequencing cost by eliminating the need to perform standard whole genome sequencing (WGS).

In accordance with the above embodiments, methods of the present disclosure include the use of cfTAPS to generate information pertaining to methylation signatures, methylation biomarkers, DNA fragment profiles, DNA sequence information (e.g., variants), and tissue-of-origin information in a single experiment to diagnose/detect cancer in a subject. As would be recognized by one of ordinary skill in the art based on the present disclosure, cfTAPS as disclosed herein can be used to generate any combination of methylation signatures, methylation biomarkers, DNA fragment profiles, DNA sequence information (e.g., variants), and tissue-of-origin information to diagnose/detect cancer in a subject. In some embodiments, a methylation signature can be obtained, and one or more of a methylation biomarker, a DNA fragment profile, DNA sequence information (e.g., variants), and tissue-of-origin information can also be obtained and used to diagnose/detect cancer in a subject. In some embodiments, the methylation status of a biomarker can be obtained, and one or more of a methylation signature, a DNA fragment profile, DNA sequence information (e.g., variants), and tissue-of-origin information can also be obtained and used to diagnose/detect cancer in a subject. In some embodiments, a DNA fragmentation profile can be obtained, and one or more of a methylation signature, a methylation biomarker, DNA sequence information (e.g., variants), and tissue-of-origin information can also be obtained and used to diagnose/detect cancer in a subject. In some embodiments, a DNA sequence variant can be identified, and one or more of a methylation signature, a methylation biomarker, a DNA fragment profile, and tissue-of-origin information can also be obtained and used to diagnose/detect cancer in a subject. In some embodiments, tissue-of-origin information can be obtained (e.g., from a whole genome cfDNA methylation signature), and one or more of the methylation signature, a methylation biomarker, a DNA fragment profile, and DNA sequence information (e.g., variants), can also be obtained and used to diagnose/detect cancer in a subject.

Accordingly, in some preferred embodiments, the present invention provides multimodal methods of analyzing cfDNA in a patient sample comprising: isolating cfDNA from a patient sample; converting 5mC and/or 5hmC residues in the sample to DHU residues to provide a modified cfDNA sample; sequencing the modified cfDNA sample to identify methylated regions in the sample, wherein a cytosine (C) to thymine (T) transition or a cytosine (C) to DHU transition in the modified cfDNA sample as compared to an unmodified reference cfDNA provides the location of either a 5mC or 5hmC in the cfDNA; and performing one or more additional analytical steps on the modified cfDNA selected from the group consisting of:
a) determining copy number variation of one or more targets in the modified cfDNA sample;
b) determining the tissue of origin or one or more targets in the modified cfDNA sample;
c) determining the fragmentation profile of the modified cfDNA sample; and
d) identifying one or more single nucleotide mutations in the modified cfDNA sample.

In some preferred embodiments, the one or more additional step is step a. In some preferred embodiments, the one or more additional step is step b. In some preferred embodiments, the one or more additional step is step c. In some preferred embodiments, the one or more additional step is step d.

In some preferred embodiments, the one or more additional steps is steps a and b. In some preferred embodiments, the one or more additional steps is step a and c. In some preferred embodiments, the one or more additional steps is steps a and d. In some preferred embodiments, the one or more additional steps is steps b and c. In some preferred embodiments, the one or more additional steps is steps b and d. In some preferred embodiments, the one or more additional steps is steps c and d.

In some preferred embodiments, the one or more additional steps is steps a, b and c. In some preferred embodiments, the one or more additional steps is steps a, b and d. In some preferred embodiments, the one or more additional steps is steps b, c and d.

In some preferred embodiments, the one or more additional steps are all of steps a, b, c and d.

In some embodiments, an unmodified reference cfDNA to be compared to a modified cfDNA sample may comprise any unmodified reference cfDNA, including for instance, a publicly available reference cfDNA or an unmodified control sample from the patient.

In some embodiments, performing TAPS on the sequencing library to obtain the whole-genome methylation signature comprises identifying 5mC modifications in the cfDNA and providing a quantitative measure for frequency of the 5mC modifications. In some embodiments, performing TAPS on the sequencing library to obtain the whole-genome methylation signature comprises identifying 5hmC modifications in the cfDNA and providing a quantitative measure for frequency of the 5hmC modifications. In some embodiments, performing TAPS on the sequencing library to obtain the whole-genome methylation signature comprises identifying 5caC modifications in the cfDNA and providing a quantitative measure for frequency of the 5caC modifications. In some embodiments, performing TAPS on the sequencing library to obtain the whole-genome methylation signature comprises identifying 5fC modifications in the cfDNA and providing a quantitative measure for frequency of the 5fC modifications.

As would be recognized by one of ordinary skill in the art based on the present disclosure, the methods described herein (e.g., cfTAPS) can be used to diagnose/detect any type of cancer. Types of cancers that can be detected/diagnosed using the methods of the present disclosure include, but are not limited to, lung cancer, melanoma, colon cancer, colorectal cancer, neuroblastoma, breast cancer, prostate cancer, renal cell cancer, transitional cell carcinoma, cholangiocarcinoma, brain cancer, non-small cell lung cancer, pancreatic cancer, liver cancer, gastric carcinoma, bladder cancer, esophageal cancer, mesothelioma, thyroid cancer, head and neck cancer, osteosarcoma, hepatocellular carcinoma, carcinoma of unknown primary, ovarian carcinoma, endometrial carcinoma, glioblastoma, Hodgkin lymphoma and non-Hodgkin lymphomas. In some embodiments, types of cancers or metastasizing forms of cancers that can be detected/diagnosed by the methods of the present disclosure include, but are not limited to, carcinoma, sarcoma, lymphoma, germ cell tumor and blastoma. In some embodiments, the cancer is invasive and/or metastatic cancer (e.g., stage II cancer, stage III cancer or stage IV cancer). In some embodiments, the cancer is an early stage cancer (e.g., stage 0 cancer, stage I cancer), and/or is not invasive and/or metastatic cancer.

In some embodiments, the methods of the present disclosure (e.g., cfTAPS) can be used to determine whether a subject has hepatocellular carcinoma (HCC) or pancreatic ductal adenocarcinoma (PDAC). In some embodiments, the method includes determining whether a subject has early stage hepatocellular carcinoma (HCC) or early stage pancreatic ductal adenocarcinoma (PDAC).

In accordance with these embodiments, the present disclosure provides methods for identifying the location of one or more of 5mC, 5hmC, 5caC and/or 5fC in a nucleic acid quantitatively with base-resolution without affecting the unmodified cytosine. In some embodiments, the nucleic acid is DNA. In some embodiments, the DNA is cfDNA (e.g., circulating cfDNA). In some embodiments, the nucleic acid is RNA. In some embodiments, a nucleic acid sample comprises a target nucleic acid that is DNA or a target nucleic acid that is RNA. In some embodiments, the methods are applied to a whole genome, and not limited to a specific target nucleic acid.

The nucleic acid may be any nucleic acid having cytosine modifications (i.e., 5mC, 5hmC, 5fC, and/or 5caC). The nucleic acid can be a single nucleic acid molecule in the sample, or may be the entire population of nucleic acid molecules in a sample (whole genome or a subset thereof). The nucleic acid can be the native nucleic acid from the source (e.g., cells, tissue samples, etc.) or can pre-converted into a high-throughput sequencing-ready form, for example by fragmentation, repair and ligation with adapters for sequencing. Thus, nucleic acids can comprise a plurality of nucleic acid sequences such that the methods described herein may be used to generate a library of target nucleic acid sequences that can be analyzed individually (e.g., by determining the sequence of individual targets) or in a group (e.g., by high-throughput or next generation sequencing methods).

A nucleic acid sample can be obtained from an organism from the Monera (bacteria), Protista, Fungi, Plantae, and Animalia Kingdoms. Nucleic acid samples may be obtained from a from a patient or subject, from an environmental sample, or from an organism of interest. In some embodiments, the sample is obtained from a human subject/patient, including but not limited to, a human with cancer or a human suspected of having cancer. In some embodiments, the sample is obtained from a tissue or cell from a human (e.g., obtained from a biopsy), including a tissue or cell that is cancerous or suspected of being cancerous. In some embodiments, the nucleic acid sample is extracted or derived from a cell or collection of cells, a bodily fluid, a tissue sample, an organ, and an organelle. In some embodiments, the nucleic acid sample is obtained from a bodily fluid, including but not limited to, blood (plasma, serum, whole blood), urine, feces/fecal fluid, semen (seminal fluid), vaginal secretions, cerebrospinal fluid (CSF), ascitic fluid, synovial fluid, pleural fluid (pleural lavage), pericardial fluid, peritoneal fluid, amniotic fluid, saliva, nasal fluid, otic fluid, gastric fluid, breast milk, and any other bodily fluid comprising cfDNA, as well as cell culture supernatants. In some embodiments, the sample is obtained from a bodily fluid that is cancerous or suspected of being cancerous. Because the methods of the present disclosure utilize mild enzymatic and chemical reactions that avoid the substantial degradation of nucleic acids associated with methods like bisulfite sequencing, the methods of the present disclosure are useful in analysis of low-input samples, such as circulating cell-free DNA and in single-cell analysis.

In some embodiments, the DNA sample comprises picogram quantities of DNA. In some embodiments, the DNA sample comprises from about 1 μg to about 900 pg DNA, from about 1 μg to about 500 pg DNA, from about 1 μg to about 100 pg DNA, from about 1 μg to about 50 pg DNA, or from about 1 to about 10 pg DNA. In some embodiments, the DNA sample comprises less than about 200 pg, less than about 100 pg DNA, less than about 50 pg DNA, less than about 20 pg DNA, less than about 15 pg DNA, less than about 10 pg DNA, or less than about 5 pg DNA.

In some embodiments, the DNA sample comprises nanogram quantities of DNA. The sample DNA for use in the methods of the present disclosure can be any quantity including, but not limited to, DNA from a single cell or bulk DNA samples. In some embodiments, the methods can be performed on a DNA sample comprising from about 1 to about 500 ng of DNA, from about 1 to about 200 ng of DNA, from about 1 to about 100 ng of DNA, from about 1 to about 50 ng of DNA, from about 1 to about 10 ng of DNA, from about 2 to about 5 ng of DNA. In some embodiments, the DNA sample comprises less than about 100 ng of DNA, less than about 50 ng of DNA, less than 40 ng of DNA, less than 30 ng of DNA, less than 20 ng of DNA, less than 15 ng of DNA, less than 5 ng of DNA, and less than 2 ng of DNA. In some embodiments, the DNA sample comprises microgram quantities of DNA.

A DNA sample used in the methods described herein may be from any source including, for example a bodily fluid, tissue sample, organ, organelle, cell or collection of cells. In some embodiments, the DNA sample is obtained from a human subject/patient, including but not limited to, a human with cancer or a human suspected of having cancer. In some embodiments, the DNA sample is obtained from a tissue or cell from a human (e.g., obtained from a biopsy), including a tissue or cell that is cancerous or suspected of being cancerous. In some embodiments, the DNA sample is extracted or derived from a cell or collection of cells, a bodily fluid, a tissue sample, an organ, and an organelle. In some embodiments, the DNA sample is obtained from a bodily fluid, including but not limited to, blood (plasma, serum, whole blood), urine, feces/fecal fluid, semen (seminal fluid), vaginal secretions, cerebrospinal fluid (CSF), ascitic fluid, synovial fluid, pleural fluid (pleural lavage), pericardial fluid, peritoneal fluid, amniotic fluid, saliva, nasal fluid, otic fluid, gastric fluid, breast milk, and any other bodily fluid comprising cfDNA, as well as cell culture supernatants. In some embodiments, the DNA sample is obtained from a bodily fluid that is cancerous or suspected of being cancerous. In some embodiments, the DNA sample is circulating cell-free DNA (cell-free DNA or cfDNA), which is DNA found in the blood and is not present within a cell. As would be recognized by one of ordinary skill in the art based on the present disclosure, cfDNA can be isolated from a bodily fluid using methods known in the art. Commercial kits are available for isolation of cfDNA including, for example, the Circulating Nucleic Acid Kit (Qiagen). The DNA sample may result from an enrichment step, including, but is not limited to antibody immunoprecipitation, chromatin immunoprecipitation, restriction enzyme digestion-based enrichment, hybridization-based enrichment, or chemical labeling-based enrichment.

The DNA may be any DNA having cytosine modifications (i.e., 5mC, 5hmC, 5fC, and/or 5caC) including, but not limited to, DNA fragments and/or genomic DNA. The DNA can be a single DNA molecule in the sample, or may be the entire population of DNA molecules in a sample (whole genome or a subset thereof). The DNA can be the native DNA from the source or pre-converted into a high-throughput sequencing-ready form, for example by fragmentation, repair and ligation with adapters for sequencing. Thus, DNA can comprise a plurality of DNA sequences such that the methods described herein may be used to generate a library of target DNA sequences that can be analyzed individually (e.g., by determining the sequence of individual targets) or in a group (e.g., by high-throughput or next generation sequencing methods).

In accordance with these embodiments, the methods of the present disclosure include the step of converting the 5mC and 5hmC (or just the 5mC if the 5hmC is blocked) to 5caC and/or 5fC. In some embodiments, this step comprises contacting the DNA or RNA sample with a ten eleven translocation (TET) enzyme. The TET enzymes are a family of enzymes that catalyze the transfer of an oxygen molecule to the C5 methyl group on 5mC resulting in the formation of 5-hydroxymethylcytosine (5hmC). TET further catalyzes the oxidation of 5hmC to 5fC and the oxidation of 5fC to form 5caC. TET enzymes useful in the methods of the present disclosure include one or more of human TET1, TET2, and TET3; murine TET1, TET2, and TET3; Naegleria TET (NgTET); Coprinopsis cinerea (CcTET); the catalytic domain of mouse TET1 (mTET1CD); and derivatives or analogues thereof. In some embodiments, the TET enzyme is NgTET. In some embodiments, the TET enzyme is human TET1 (hTET1). In some embodiments, the TET enzyme is mTET1CD.

Methods of the present disclosure can also include the step of converting the 5caC and/or 5fC in a nucleic acid sample to DHU. In some embodiments, this step comprises contacting the DNA or RNA sample with a reducing agent including, for example, a borane reducing agent such as pyridine borane, 2-picoline borane (pic-BH₃), borane, sodium borohydride, sodium cyanoborohydride, and sodium triacetoxyborohydride. In some embodiments, the reducing agent is pyridine borane and/or pic-BH₃.

The methods of the present disclosure can also include the step of amplifying the copy number of a modified nucleic acid by methods known in the art. When the modified nucleic acid is DNA, the copy number can be increased by, for example, PCR, cloning, and primer extension. The copy number of individual target DNAs can be amplified by PCR using primers specific for a particular target DNA sequence. Alternatively, a plurality of different modified target DNA sequences can be amplified by cloning into a DNA vector by standard techniques. In some embodiments, the copy number of a plurality of different modified target DNA sequences is increased by PCR to generate a library for next generation sequencing where, e.g., double-stranded adapter DNA has been previously ligated to the sample DNA (or to the modified sample DNA) and PCR is performed using primers complimentary to the adapter DNA.

In some embodiments, the method comprises the step of detecting the sequence of the modified nucleic acid. The modified target DNA or RNA contains DHU at positions where one or more of 5mC, 5hmC, 5fC, and 5caC were present in the unmodified target DNA or RNA. DHU acts as a T in DNA replication and sequencing methods. Thus, the cytosine modifications can be detected by any direct or indirect method that identifies a C to T transition known in the art. Such methods include sequencing methods such as Sanger sequencing, microarray, and next generation sequencing methods. The C to T transition can also be detected by restriction enzyme analysis where the C to T transition abolishes or introduces a restriction endonuclease recognition sequence.

Embodiments of the present disclosure also provide kits for identification of 5mC and 5hmC in a DNA. Such kits comprise reagents for identification of 5mC and 5hmC by the methods described herein. The kits may also contain the reagents for identification of 5caC and for the identification of 5fC by the methods described herein. In some embodiments, the kit comprises a TET enzyme, a borane reducing agent and instructions for performing the method. In some embodiments, the TET enzyme is TET1 and the borane reducing agent is selected from one or more of the group consisting of pyridine borane, 2-picoline borane (pic-BH3), borane, sodium borohydride, sodium cyanoborohydride, and sodium triacetoxyborohydride. In some embodiments, the TET1 enzyme is NgTet1 or murine Tet1 (e.g., mTet1CD) and the borane reducing agent is pyridine borane and/or pic-BH₃.

In some embodiments, the kit further comprises a 5hmC blocking group and a glucosyltransferase enzyme. In some embodiments, the blocking group added to 5hmC is a sugar. In some embodiments, the sugar is a naturally-occurring sugar or a modified sugar, for example glucose or a modified glucose. In some embodiments, the blocking group is added to 5hmC by contacting a nucleic acid sample with UDP linked to a sugar, for example UDP-glucose or UDP linked to a modified glucose in the presence of a glucosyltransferase enzyme, for example, T4 bacteriophage β-glucosyltransferase (βGT) and T4 bacteriophage α-glucosyltransferase (αGT) and derivatives and analogs thereof.

In some embodiments, the kit further comprises an oxidizing agent selected from potassium perruthenate (KRuO4) and/or Cu(II)/TEMPO (copper(II) perchlorate and 2,2,6,6-tetramethylpiperidine-1-oxyl (TEMPO)). In some embodiments, the kit comprises reagents for blocking 5fC in the nucleic acid sample. In some embodiments, the kit comprises an aldehyde reactive compound including, for example, hydroxylamine derivatives, hydrazine derivatives, and hydrazide derivatives as described herein. In some embodiments, the kit comprises reagents for blocking 5caC as described herein. In some embodiments, the kit comprises reagents for isolating DNA or RNA. In some embodiments the kit comprises reagents for isolating low-input DNA from a sample, for example cfDNA from blood, plasma, or serum.

In some embodiments, the methods of the present disclosure include treating a patient (e.g., a patient with cancer, with early-stage cancer, or who is suspected of having cancer). In some embodiments, the methods includes determining a methylation signature as provided herein and administering a treatment to a patient based on the results of determining the methylation signature. The treatment can include administration of a pharmaceutical compound, a vaccine, performing a surgery, imaging the patient, and/or performing another test. In some embodiments, the methods of the present disclosure can be used as part of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, methods of the present disclosure include diagnosing cancer in a subject. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example a methylation biomarker and/or a methylation signature, which is indicative of the presence, severity, or absence of the condition (e.g., cancer).

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment of a subject based on methylation signature can be useful to separate subjects with good prognosis and/or low risk of developing cancer who will need no therapy or limited therapy from those more likely to develop cancer or suffer a recurrence of cancer who might benefit from more intensive treatments. As such, “making a diagnosis” or “diagnosing”, as used herein, is further inclusive of making a determination of a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the identification and assessment of a methylation signature, as disclosed herein.

In some embodiments, methods of the present disclosure include determining whether to initiate or continue prophylaxis or treatment of a cancer in a subject. In some embodiments, the method comprises providing a series of biological samples over a time period from the subject; analyzing the series of biological samples to determine a methylation signature as disclosed herein in each of the biological samples; and comparing any measurable change in the methylation signatures in each of the biological samples. Any changes in the methylation signatures over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation signatures can be measured in each of the samples taken from different time points and qualitative and/or quantitative differences noted. A change in the methylation signatures from the different samples can be correlated with risk for developing cancer, prognosis, determining treatment efficacy, and/or progression of the cancer in the subject. In some embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 4. MATERIALS AND METHODS

Experimental design. Whole blood samples from 30 non-cancer controls were obtained from John Radcliffe hospital (Ethical approvals IDs 16/YH/0247 and 18/WM/0237). Pancreatitis blood samples from 8 patients were obtained from John Radcliffe hospital. The study was approved by Oxfordshire REC-A (10/H0604/51) and is registered on the UK NIHR portfolio as study number 10776. PDAC patients were consented for this study via the Oxford Radcliffe Biobank (09/H0606/5+5, project: 19/A177) and whole-blood samples were collected from 24 patients. Collection of plasma samples from 21 HCC and 4 cirrhosis patients was REC approved (Ethical approval 2/NE/0395, IRAS project ID:116370). No sample-size calculations were performed. Sample size was determined based on availability. PDAC, HCC, pancreatitis and cirrhosis samples were collected from subjects with clinically diagnosed disease. Non-cancer control samples were collected from individuals without cancer diagnosis at the time of sample collection or previous history of cancer.

The main goal of the study was comprehensive, multidimensional characterization of cfDNA in cancer and controls by whole-genome methylation sequencing using TAPS. CfDNA TAPS libraries were constructed and paired-end 150 bp sequenced on aNovaSeq 6000 sequencer (Illumina). Technical details are described in the sections below. Samples with 5mC conversion below 90% calculated based on methylated lambda spike-in control were excluded from downstream analysis.

Collection and preparation of cfDNA samples. Blood was collected into EDTA-coated Vacutainers. Plasma was separated from collected blood samples within 4 h from collection. Plasma was collected by centrifuging blood at 1600×g for 10 min at 4° C. and 16000×g for 10 min at 4° C. and stored at −80° C. for cfDNA purification. cfDNA from plasma was extracted using Qiamp Circulating Nuclei Acid Kit (Qiagen). cfDNA was quantified by Qubit Fluorometer (Life Technologies).

Preparation of carrier DNA and spike-in controls. Carrier DNA was prepared by PCR amplification of the pNIC28-Bsa4 plasmid (Addgene, cat. no. 26103) in a reaction containing 1 ng DNA template, 0.5 pM primers (Fwd: 5′-AGGCAACTTTATGCCCATGCAA-3′ (SEQ ID NO: 2); Rev: 5′-CCAAGGGGTTATGCTAGTTATTGC-3′ (SEQ ID NO: 3)) and 1× Phusion High-Fidelity PCR Master Mix with HF Buffer (Thermo Scientific). The CpG-methylated lambda DNA and 2 kb unmodified spike-in control DNA were prepared as described previously. CpG-methylated lambda DNA, carrier DNA and 2 kb unmodified control were fragmented by Covaris M220 (Peak Incident Power—50 W, Duty Factor—20%, Cycles per Burst (cpb)—200, time—150 s) and size-selected on 0.9--1.2×AMPure XP beads to select for 150-250 bp fragments.

38| Preparation of sequencing adapters. Adapter oligos (5′-ACACTCTTTCCCTACACGACGCTCTTCCGATCT-3′ (SEQ ID NO: 4); 5′-/5Phos/GATCGGAAGAGCACACGTCT-3′ (SEQ ID NO: 5)) were obtained from IDT with HPLC purification. Adapter oligos were annealed together in a 50 μL reaction containing 15 μM of each oligo, 10 mM Tris-Cl (pH=8.0), 0.1 mM EDTA (pH=8.0) and 50 mM NaCl with the following program: 2 min at 95° C., 140 cycles of 20 sec at 95° C. (decrease temperature 0.5° C. every cycle) and hold at 4° C. Annealed 15 μM Illumina multiplexing adapters were then aliquoted into small single-use vials and stored at −80° C.

mTet1CD oxidation. mTet1CD was prepared as described previously. DNA was incubated in a 50 μl reaction containing 50 mM HEPES buffer (pH 8.0), 100 μM ammonium iron (II) sulfate, 1 mM α-ketoglutarate, 2 mM ascorbic acid, 2 mM dithiothreitol, 100 mM NaCl, 1.2 mM ATP and 4 μM mTet1CD for 80 min at 37° C. After that, 0.8 U of Proteinase K (New England Biolabs) were added to the reaction mixture and incubated for 1 h at 50° C. The product was cleaned up on Bio-Spin P-30 Gel Column (Bio-Rad) and 1.8×AMPure XP beads following the manufacturer's instruction.

Pyridine borane reduction. Oxidized DNA in 35 μl of water was reduced in a 50 μl reaction containing 600 mM sodium acetate solution (pH 4.3) and 1 M pyridine borane (Alfa Aesar) for 16 h at 37° C. and 850 r.p.m. in an Eppendorf ThermoMixer. The product was purified using Zymo-Spin columns.

cfDNA TAPS. 10 ng of cfDNA were spiked-in with 0.15% CpG-methylated lambda DNA and 0.015% unmodified 2 kb control and used for an end-repair and A-tailing reaction and ligated to Illumina Multiplexing adapters with KAPA HyperPrep kit according to the manufacturer's protocol. Subsequently 100 ng of carrier DNA were added to ligated libraries and samples were double-oxidized with mTet1CD and reduced with pyridine borane according as described above. Converted libraries were amplified using NEBNext® Multiplex Oligos for Illumina® (96 Unique Dual Index Primer Pairs) with KAPA Hifi Uracil Plus Polymerase for 7 cycles and cleaned up on 1×AMPure XP beads. CfDNA TAPS libraries were paired-end 150 bp sequenced on a NovaSeq 6000 sequencer (Illumina).

TAPS mapping and pre-processing. Raw sequenced reads were processed with trim_galore (version 0.6.2 www.bioinformatics.babraham.ac.uk/projects/trim_galore/) to trim adapter and low-quality bases with the following parameters --paired --length 35 --gzip --cores 2. Clean reads were aligned to human reference genome (GRCh38 ftp.ncbi.nlm.nih.gov/genomes/all/GCA/000/001/405/GCA_000001405.15_GRCh38/seqs_for alignment_pipelines.ucsc_ids/GCA_000001405.15_GRCh38_no_alt analysis_set.fna.gz.) combining spike-in sequences using bwa mem (version 0.7.17-r1188) with the following parameters -I 500,120,1000,20. Reads with MAPQ<1 were excluded from further analysis. Picard MarkDuplicates (version 2.18.29-SNAPSHOT) was used to identify duplicate reads. MethylDackel extract (version 0.5.0 https://github.com/dpryan79/MethylDackel) was used for methylation calling using the following parameters -q 10 -p 13 -t 4 --mergeContext --OT 10,140,75,75 --OB 10,140,75,75. CpG sites overlapped with common SNP (dbSNP153), blacklisted regions, centromeres, and sex chromosomes were excluded for further analysis.

cfDNA WGBS analysis. CfDNA WGBS data was downloaded from EGAD00001004317. Raw sequenced reads were processed with trim_galore (version 0.6.2 www.bioinformatics.babraham.ac.uk/projects/trim_galore): adapter and low-quality bases were trimmed with the following parameters --paired --length 35 --gzip --cores 2. Clean reads were aligned to human reference genome (GRCh38) using bismark (Bismark Version: v0.22.0) with default parameters. deduplicate_bismark was used for deduplication. Samtools was used to filter the fragments with -q 10, and only reads mapped in proper pairs were used for fragmentation analysis. bismark_methylation_extractor was used to extract methylation from deduplicated bam files with default parameters.

PCA on DNA methylation and feature overrepresentation analysis. The genome was binned into 1 kb windows. Methylation level was calculated using number of methylated CpGs divided by the number of total CpGs sequenced. Windows with mean CpG coverage (number of total CpG sequenced/total number of CpG positions)<2 were excluded for further analysis. Dimdesc was used with parameter proba=0.01 to determine the regions that contribute most to each principal component obtained by the PCA function (largest eigenvalues of each eigenvector). Bedtools fisher was used to test the number of overlaps between the top 200 contributing regions (sorted by absolute correlation value) and the selected genomic features. Selected genomic features included regulatory element from Ensemble (ftp.ensembl.org/pub/release-97/regulation/homo_sapiens/homo_sapiens.GRCh38.Regulatory_Build.regulatory_features.2 0190329.gff.gz) and CpG islands from UCSC (hgdownload.soe.ucsc.edu/goldenPath/hg38/database/cpgIslandExt.txt.gz).

Two class prediction using DNA methylation signature. Two class prediction models were trained and evaluated based on a LOO approach. Briefly, one sample was held out as the testing set while the remaining samples were used for model training. DMRs (promoters for PDAC and enhancers for HCC) were identified in the training set by t-test (P value<0.002, methylation difference>0.05). In each leave-one-out fold 443-775 differentially methylated enhancers and 160-318 differentially methylated promoters were identified in the HCC vs. non-cancer control and PDAC vs. non-cancer control feature selection steps, respectively. In total, 1,521 enhancers, and 531 promoters were selected during the cross-validation process. The predictive model was built on selected DMRs using cv. Glmnet and validated on the test sample. This procedure was repeated N times, where N=number of samples. ROC curves were prepared in R based on the predicted scores of held out test samples from cvglm models. Cirrhosis patients and cfDNA WGBS data were used as independent validation sets to evaluate the performance of HCC model. Pancreatitis patients were used as independent validation set to evaluate the performance of PDAC model. Aligned BAM files were down-sampled from 100M to 200M read pairs using samtools view. For each down-sampled set, the method described above was used to detect DMRs. Ref DMR were defined as the total unique DMR in the LOO cross-validations. The percentage of ref DMRs were computed by dividing the overlapped DMR between down-sampled set and the ref DMR and the total ref DMR.

GO analysis ofDMRs. Genes regulated by differentially methylated enhancers in HCC cfDNA were identified using the GeneHancer database. The genes closest to the differentially methylated promoters in PDAC were identified as related using following R packages: AnnotationHub (version 2.18.0), TxDb.Hsapiens.UCSC.hg38.knownGene (version 3.10.0) and org.Hs.eg.db (version 3.10.0). GO analysis was performed on these identified genes using Enrichr tool against NCI-Nature Pathway Interaction database.

Tissue Reference Map. CpG-level tissue methylation data was collated from six public sources (sources of public methylation WGBS data for generation of tissue map are not included in the present disclosure but can be made available upon request). After filtering diseased, sex-specific, and low-coverage samples, 144 healthy, adult tissue samples were retained, and grouped into 32 physiologically distinct tissue groups (raw data pertaining to cfDNA tissue contribution for each patient in cfTAPS cohort are not included in the present disclosure but can be made available upon request). 133 out of 144 samples were already aligned to hg38; the remaining 11 samples were converted from hg19 to hg38 using the UCSC hgLiftOver tool.

About 79,000 enhancers were filtered from Ensembl Regulatory Build using a tissue-specific DMR finding algorithm similar to Moss et al. Specifically, this algorithm performs pairwise one-vs-all comparisons for each tissue group in the reference atlas, selecting the regions which show the largest median methylation difference and consistent methylation across the tissue group in question. As in Moss et al., pairwise tissue group correlations were also calculated, and included DMRs that best separated each tissue group from the first and second most highly correlated tissue.

Tissue Deconvolution by Non-negative Least Squares Regression. Tissue deconvolution was performed using non-negative least squares regression and implemented using Scipy's optimize function in Python 3.8. Given a tissue reference matrix A, and a vector of observed methylation ratios yₛ in a sample s, the tissue contribution x was estimated by solving the following minimization problem:

$$min {‖ Ax - {y}_{s} ‖}^{2}$$

$$subject to x \geq 0.$$

Fragmentation analysis. The length of the DNA fragments was obtained from alignment files using Samtools. Fragmentation profiles were calculated as the fraction of cfDNA fragments at 10 bp length range bins. PCA analysis and plots were generated in R.

For fragmentation-based prediction, proportion of cfDNA fragments (300 to 500 bp) in 10 bp length range bins was calculated. Models were built and trained by leave-one-out approach using cv. glmnet method. ROC curves were prepared in R based on prediction scores from validation.

CNV analysis. Alignment files for each sample were downsampled to 225M read pairs with samtools view. QDNAseq package was used for copy number variation analysis. The bin annotation was downloaded from QDNAseq.hg38 (github.com/asntech/QDNAseq.hg38) and bin size 100 kb was used. Regions which were blacklisted or have mappability less than 80 were excluded for further analysis. cutoffs 0.8 and 1.2 were used to define copy number losses and gains respectively in the callBins function. Patients which have copy number aberrations with length range bigger than 500 kb were classified as patients with CNV.

Three class prediction models. Three class prediction models were trained and evaluated based on a LOO approach. For DNA methylation, the candidate features were initially narrow down to 824,320 1 kb windows encompassing mapping to regulatory regions as mentioned previously. The methylation model aims to capture the cancer-type specific methylation change by selecting DMRs based on a pairwise comparison using a t-test. DMRs were then ranked by P value, and the top 5 DMRs in each pairwise comparison were selected for model training. The prediction model was built on DMRs selected among the training set using a SVM model implemented in the caret package (train method=“svmLinear2”) and validated on the test sample. This procedure was repeated N times, where N=number of samples. For tissue contribution and fragmentation fraction, the raw matrixes were used to build models following the same method as for DMRs. These three models were integrated by taking the averaged (mean) predictions across the three modalities, where the selected prediction in each case was the one with the maximum averaged predicted score.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all joumal references, U.S. patents, and publications referred to herein are hereby incorporated by reference in their entireties.

The present disclosure has multiple aspects, illustrated by the following non-limiting examples.

### Example 1

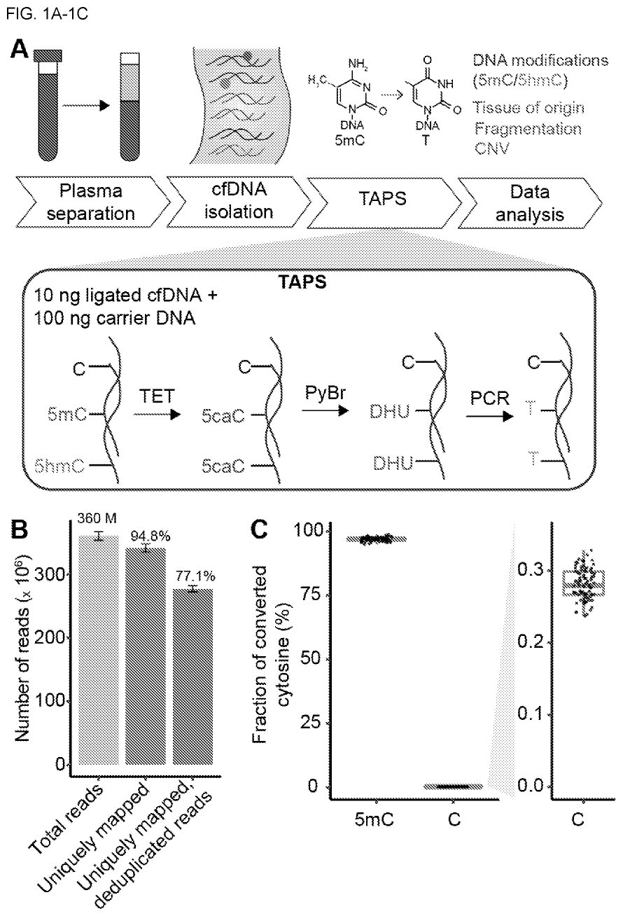

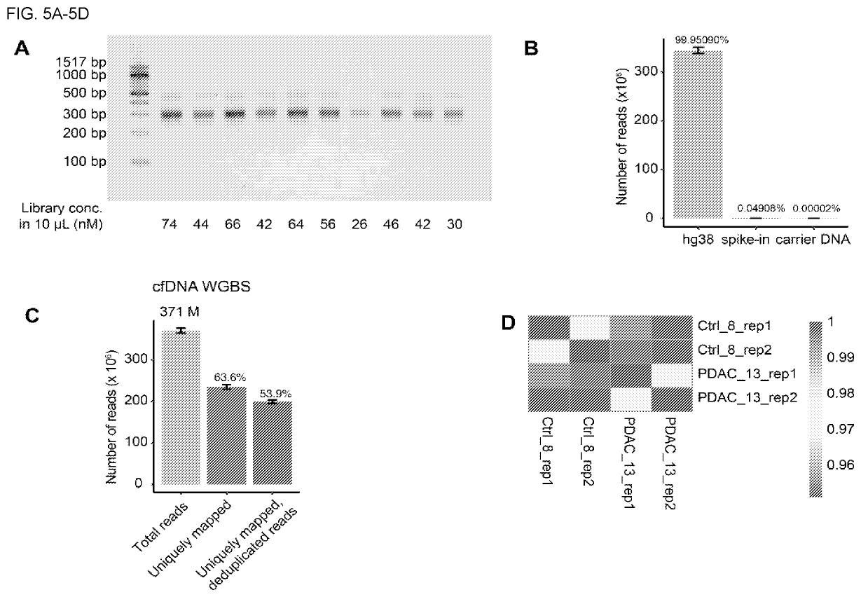

Adaptation of TAPS for cfDNA sequencing. Experiments were conducted to optimize the TAPS protocol to work with low input cfDNA (10 ng, purified from 1-3 mL of plasma). Briefly, 10 ng cfDNA is first ligated to Illumina adapters and 100 ng of carrier DNA is then added to the sample prior to TET oxidation and pyridine borane (PyBr) reduction steps (FIG. 1A). It found that the addition of carrier DNA improves the recovery of cfDNA during the workflow and results in higher library yields when compared to the standard TAPS protocol (FIG. 5A). Subsequently, 5-methylcytosine (5mC) and 5-hydroxymethylcytosine (5hmC) in cfDNA are oxidized by mTet1CD enzyme to 5-carboxylcytosine (5caC) and reduced to dihydrouracil (DHU), which is amplified as T in the final PCR step (FIG. 1A).

The cfTAPS was applied to 87 cfDNA samples. Libraries were sequenced to a mean of 360M read pairs (11.6× mean depth, range 8.2-22×), and resulted in high unique mapping rate and unique deduplicated mapping rate of 94.8% and 77.1%, respectively (FIG. 1B; raw data pertaining to sequencing statistics are not included in the present disclosure but can be made available upon request). Among the mapped reads, 99.95% were mapped to the human genome (FIG. 5B). In comparison, a recent cfDNA whole-genome bisulfite sequencing (WGBS) study sequenced to a similar depth (a mean of 371M read pairs) and resulted in significantly lower unique mapping rate (63.6%) and unique deduplicated mapping rate (53.9%) (FIG. 5C), even though it used more cfDNA input (from 5 mL plasma). This highlights the advantage of cfTAPS to generate higher quality and more complex data than cfDNA WGBS while requires less cfDNA input.

Subsequently, the accuracy of cfTAPS for detecting 5mC was assessed based on spike-in controls, which have modified and unmodified cytosines in the known positions. CpG methylated lambda DNA was used to estimate the conversion of 5mC. Two samples had a low conversion rate below 85% and were excluded from downstream analysis (raw data pertaining to sequencing statistics are not included in the present disclosure but can be made available upon request). The remaining 85 samples had a mean 5mC conversion rate of 97.0% or a false negative rate (non-conversion rate of 5mC) of 3.0% (FIG. 1C). The false positive rate (conversion rate of unmodified C), estimated based on unmodified amplicon spike-in, was 0.28%, which confirms that cfTAPS allows highly sensitive and specific detection of 5mC in cfDNA (FIG. 1C). High reproducibility of cfTAPS between technical replicates was further confirmed (FIG. 5D).

### Example 2

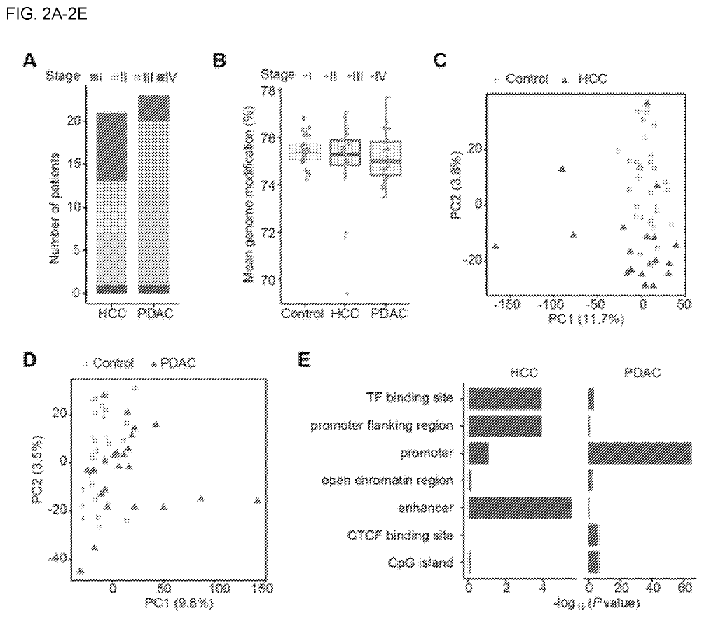

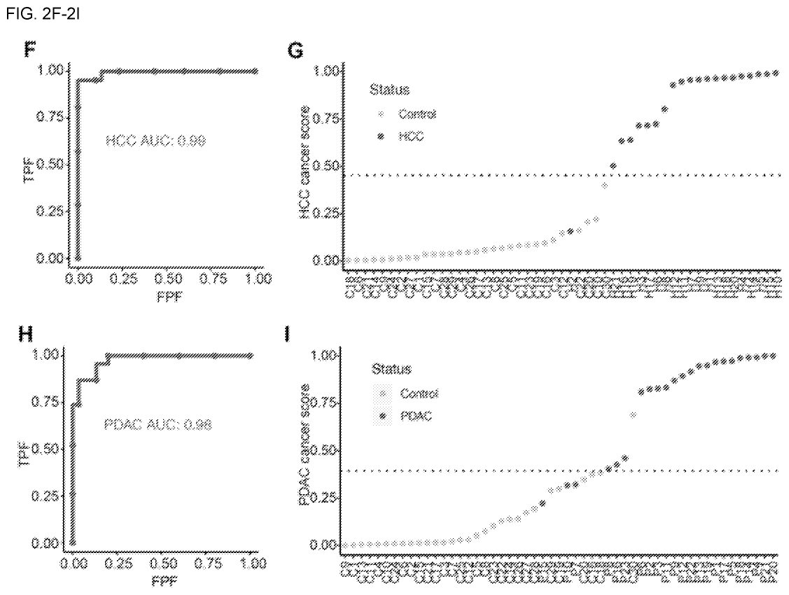

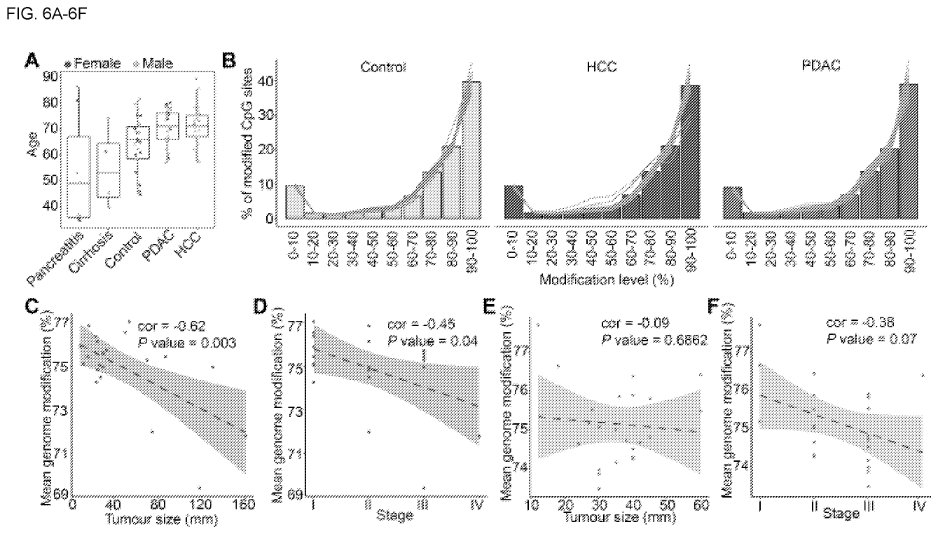

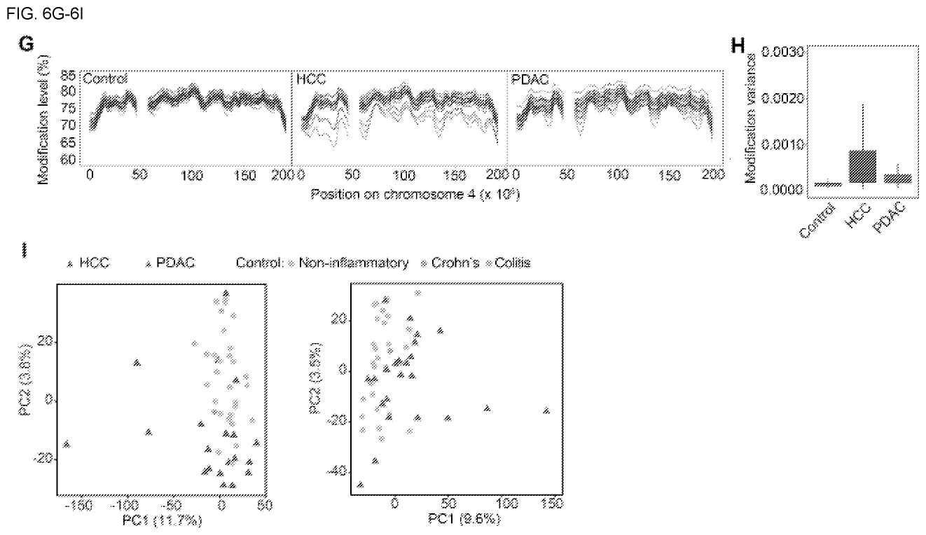

Whole-genome DNA methylation from cfTAPS. Next, experiments were conducted to characterize the cfDNA methylome in the 85 cfDNA samples that passed initial quality control. The cohort included samples from 21 patients with HCC, 23 with PDAC, 30 non-cancer controls, 4 patients with cirrhosis and 7 with pancreatitis (FIG. 6A). Cirrhosis and pancreatitis are precancerous conditions affecting liver and pancreas respectively. Most PDAC and HCC patients in the cohort were at a non-metastatic stage, with 52% of PDAC patients and 67% HCC patients at stage I and II (FIG. 2A; clinical data pertaining to the cfTAPS study cohort are not included in the present disclosure but can be made available upon request). Among the 21 HCC patients, only 4 (19%) had elevated levels of APF (over 20 ng/mL). Among the 18 PDAC patients which had CA19-9 measurement, 16 (89%) had elevated levels of CA19-9 (over 37 U/mL). However, it is important to note, that CA19-9 level is often elevated in non-malignant conditions including inflammatory disease. Of note, the non-cancer controls were collected from an endoscopy clinic and were enriched with gastro-intestinal inflammatory conditions such as Crohn's disease and colitis (clinical data pertaining to the cfTAPS study cohort are not included in the present disclosure but can be made available upon request). While distinguishing these non-cancer controls from cancer patients is more challenging than a typically healthy control group, this may provide a more real-world comparison of a diagnostic test in an aging population.

Global methylation levels of cfDNA in cancer and control samples were analyzed. CfDNA methylation displayed atypical bimodal distribution in all groups with most CpG sites either fully methylated or unmethylated (FIG. 5B). Average CpG methylation level in control samples was 75.5% and was similar in cancer cfDNA (HCC: 74.9%; PDAC: 75.1%). Previously reported global cfDNA hypomethylation in HCC was only observed in a few samples with late stage or large tumor size (FIG. 2B and FIG. 6C-6F). By contrast, a higher variance of methylation in 1 Mb genomic windows was observed between cancer patients compared to controls (FIG. 6G-6H).

Experiments were then conducted to investigate whether whole-genome cfDNA methylation signatures have the potential to discriminate between cancer patients and non-cancer controls. Principal Component Analysis (PCA) of cfDNA methylation in 1 kb genomic windows was performed first. Both HCC (FIG. 2C) and PDAC samples (FIG. 2D) showed partial separation from controls in principal component 2 (PC2) and PC1, respectively. Noted that the inflammatory patients (Crohn's disease and colitis) do not separate from the other non-cancer controls (FIG. 6I). Experiments were then conducted to investigate where the windows that most contributed to the cancer/control separation were enriched in the genome. Results indicated that the top 200 windows with the highest correlation with PC2 for HCC were enriched in enhancers (FIG. 2E). Conversely, the 200 windows most highly correlated with PC1 for PDAC were highly enriched in promoters (FIG. 2E), suggesting that different cancer types have different cfDNA methylation signals.

### Example 3

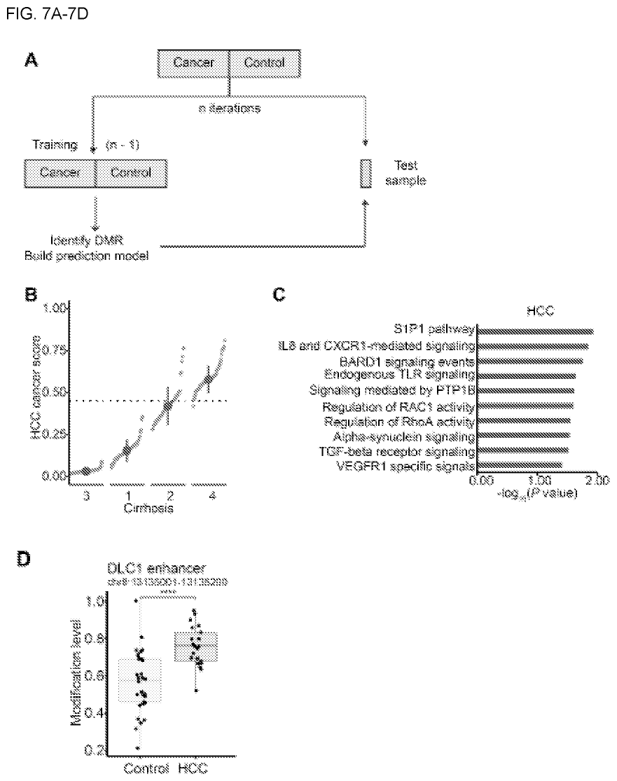

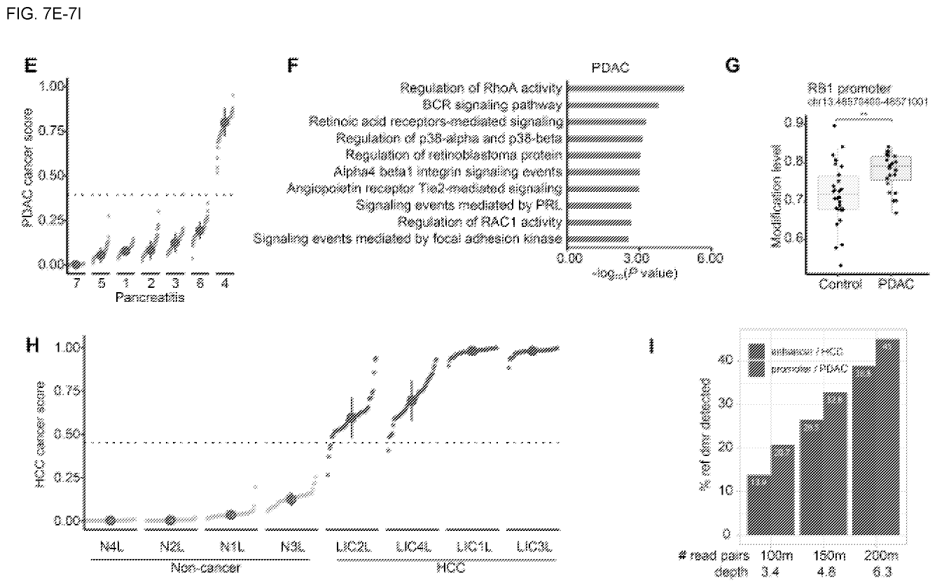

Differential DNA methylation from cfTAPS. Since methylation patterns in regulatory regions significantly contributed to discrimination between cancer and controls in unsupervised analysis, experiments were conducted to investigate the predictive potential of cfDNA methylation in enhancer and promoter regions for HCC and PDAC prediction respectively, using a supervised machine learning approach with leave-one-out (LOO) cross-validation. Briefly, in each round of LOO cross-validation, one sample was used as a validation set and the remaining samples for model training. Within each fold, differentially methylated enhancers and promoters were identified for HCC and PDAC, respectively, and used to train a regularized generalized linear model classifier (glmnet) to distinguish each cancer type from the control samples. This model was then evaluated on the held-out test sample for each fold (FIG. 7A). Cirrhosis and pancreatitis samples were not included in model building but were used as an independent validation set to evaluate performance of the classifiers to discriminate between cancer and pre-malignant conditions.

Significant prediction of HCC (AUC=0.99) was achieved based on differentially methylated enhancers (FIG. 2F-2G; raw data pertaining to differentially methylated enhancers used for HCC vs. Control predictions are not included in the present disclosure but can be made available upon request). Moreover, based on predicted scores, 3 out of 4 cirrhosis samples could be distinguished from HCC, suggesting that the model is able to detect cancer-specific features (FIG. 7B). Gene ontology analysis was then performed on the differentially methylated enhancers and found significant enrichment in signalling pathways commonly affected in liver cancer, including regulation of RAC1 activity and IL8- and CXCR1-mediated signalling (FIG. 7C). For example, in cfDNA of HCC patients, significant hypermethylation of the enhancer that regulates expression of the DLC1 gene, a tumor suppressor for human liver cancer involved in RAC1 and Rho signalling pathways, was observed (FIG. 7D).

Accurate prediction of PDAC (AUC=0.98) was achieved based on differentially methylated promoters (FIG. 2H-2I; raw data pertaining to differentially methylated promoters used for PDAC vs. Control predictions are not included in the present disclosure but can be made available upon request). Similarly, the classifier was able to predict 6 out of 7 pancreatitis samples as non-cancer, despite not being trained on any pancreatitis samples (FIG. 7E). Differentially methylated promoters in PDAC cfDNA were enriched in signalling pathways affected in PDAC including RB1 regulation and p38 signalling pathways (FIG. 7F). For instance, results indicated significant hypermethylation in the RB1 gene promoter (FIG. 7G), a well-studied tumor suppressor gene. Hypermethylation of RB1 promoter was previously found in human cancers and downregulation of RB1 were reported in pancreatic cancer.

Finally, the HCC model was validated on an independent dataset from a recent cfDNA WGBS study, which contains 4 HCC patients and 4 non-cancer controls. Results indicated that the models built on differentially methylated enhancers identified from cfTAPS data were able to correctly classify all HCC and non-cancer controls from this external dataset (FIG. 7H). It is important to note that the high sequencing depth of cfTAPS is essential for de novo differential methylation analysis from cfDNA and the differentially methylated regions (DMRs) identified were significantly decreased when the data was down-sampled to 100-200M read pairs (FIG. 7I). Taken together, cfTAPS enables whole-genome discovery of DMRs in cfDNA, and the distinct methylation patterns in regulatory regions enable accurate prediction of HCC and PDAC.

### Example 4

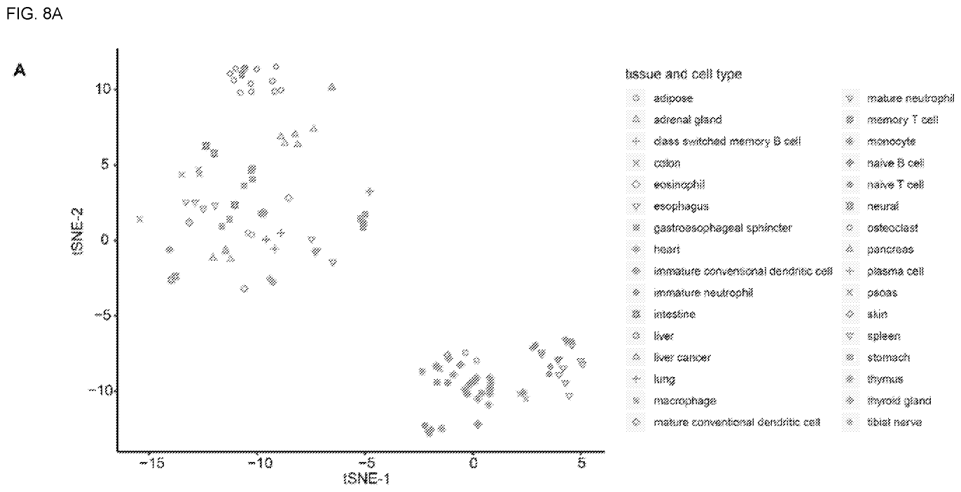

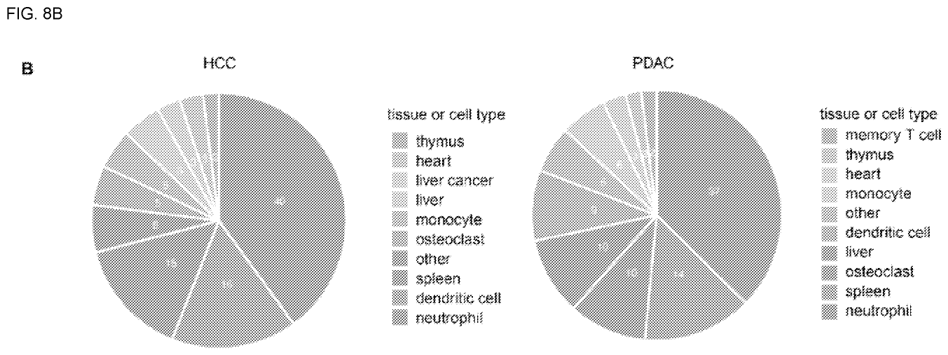

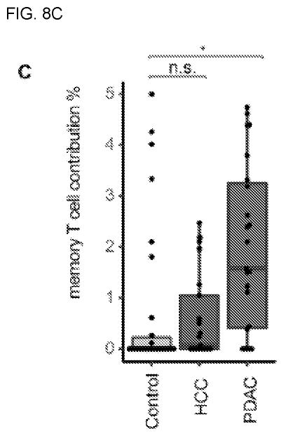

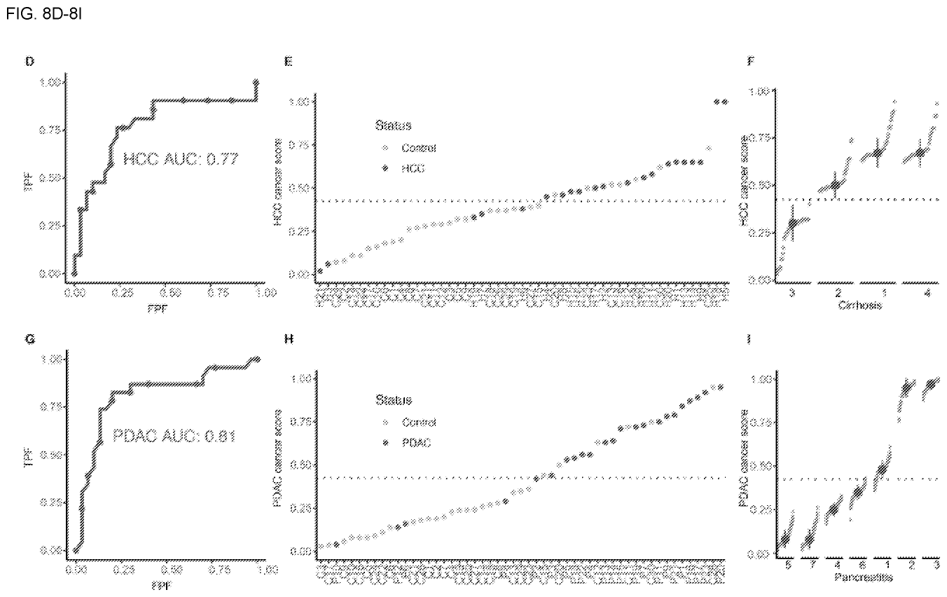

cfTAPS informs tissue-of-origin. CfDNA methylation has been shown to provide tissue-of-origin information. Most approaches use 450K methylation array tissue data, which covers less than 1% of CpGs in the human genome, to infer tissue contribution from cfDNA methylation. To further utilize the whole-genome information from cfTAPS for cfDNA deconvolution, CpG-level methylation data were collated from 144 publicly available tissue and blood cell WGBS, and stratified into 32 physiologically distinct tissue and blood cell types, including liver tumor tissue (sources of public methylation WGBS data for generation of tissue map are not included in the present disclosure but can be made available upon request). Given the prevalence of tissue-specific DNA methylation in enhancer regions, an enhancer-aggregated reference map of tissue methylation was constructed. The resulting methylation reference map displays good clustering of blood and immune cell types, and even physiologically related solid tissues (FIG. 8A).

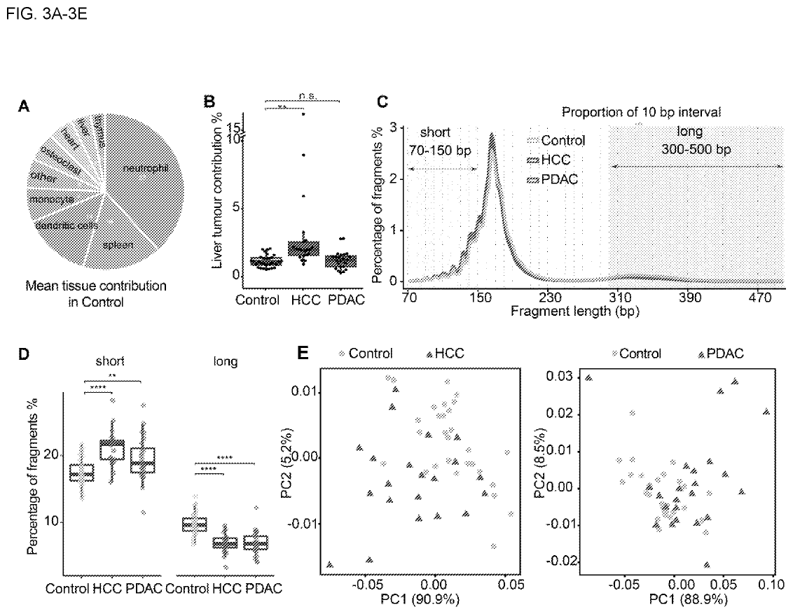

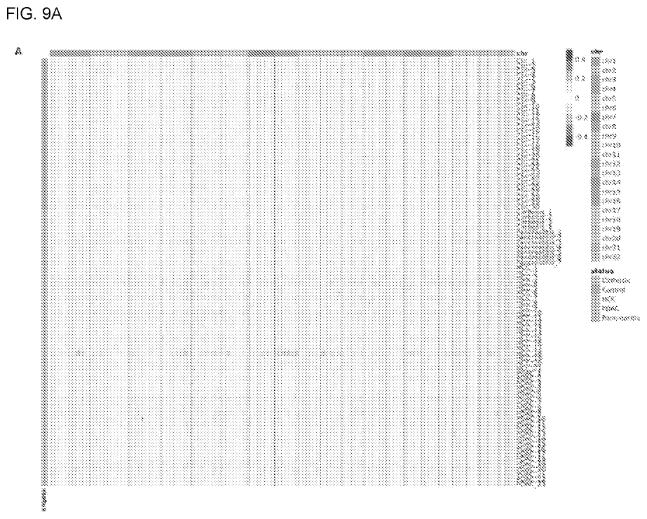

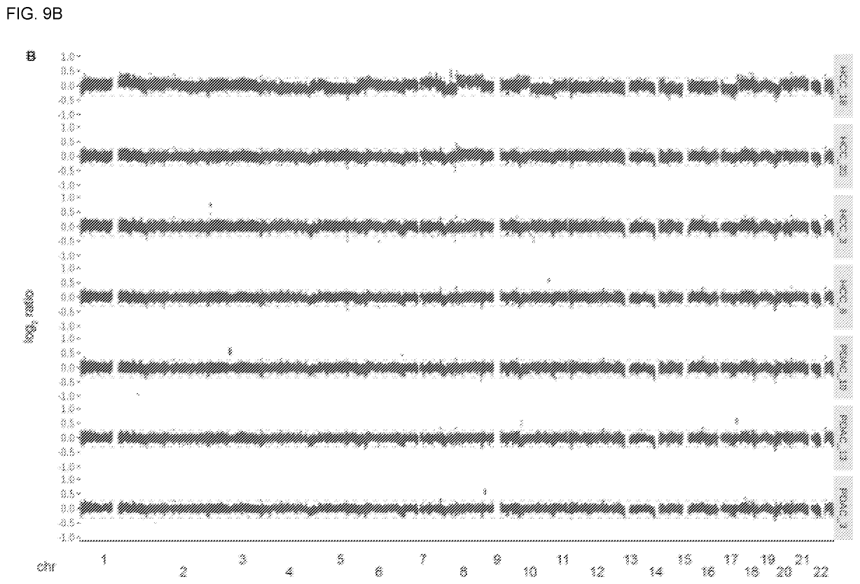

Tissue contribution in cfTAPS samples was calculated by performing non-negative least squares regression (NNLS). cfDNA tissue contribution was broadly similar between cancer and control groups, and in agreement with previous reports, with blood and immune cells dominant, and lower proportions of solid tissues (FIG. 3A, FIG. 8B; raw data pertaining to cfDNA tissue contribution for each patient in cfTAPS cohort are not included in the present disclosure but can be made available upon request). Importantly, a significantly increased liver tumor contribution in HCC alone was observed (FIG. 3B, paired t-test, P value 0.0016), and a significantly increased memory T cell contribution in PDAC samples was observed (paired t-test, P value 0.028) (FIG. 8C). A regularized generalized linear model was trained based on tissue contribution, evaluating all samples using LOO cross-validation, and was demonstrated to correctly separate the majority of samples in both cancer types (HCC vs non-cancer control: AUC=0.77; PDAC vs non-cancer control: AUC=0.81). However, these models perform worse at distinguishing pancreatitis and cirrhosis compared to methylation-based models (FIG. 9D-8I). Tissue deconvolution is currently limited by the availability of public WGBS data. Nevertheless, these results indicate that cfTAPS provides valuable tissue-of-origin information for early cancer detection.

### Example 5

Fragmentation patterns from cfTAPS. Although the main purpose of efTAPS is DNA methylation sequencing, it only induces base-changes at modified cytosines, thus keeping the majority of DNA intact. Additional genetic information can therefore be extracted from cfTAPS data to further improve the sensitivity of early cancer detection. Experiments were conducted to first investigate the CNVs from cfTAPS data. As expected with the non-advanced cancer cohort, CNVs were only predicted in 4 HCC patients and 3 PDAC patients (FIG. 9A-9B). Next, experiments were conducted to investigate whether cfTAPS can retain reliable cfDNA fragmentation information, which has recently been shown to change significantly during cancer development and has therefore been adopted in cancer detection assays.

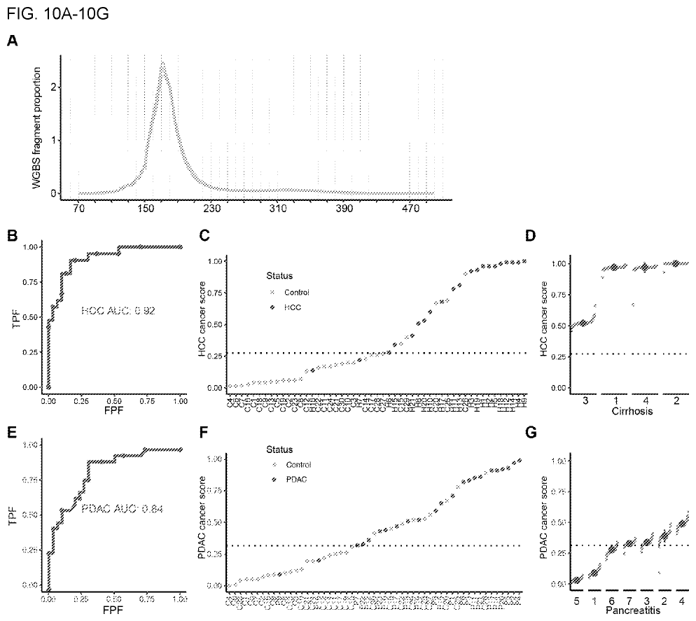

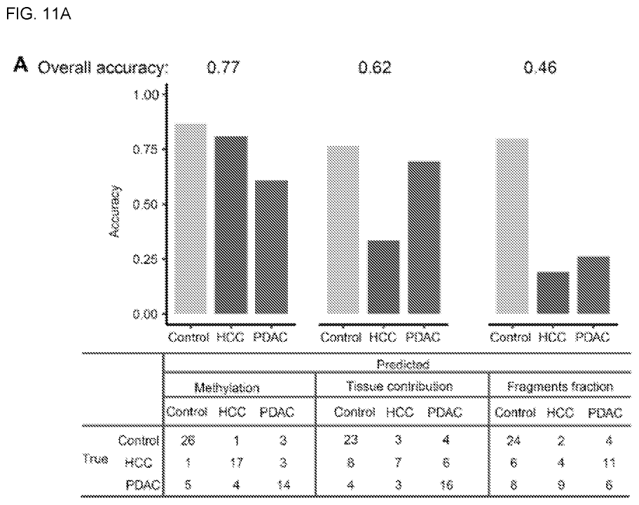

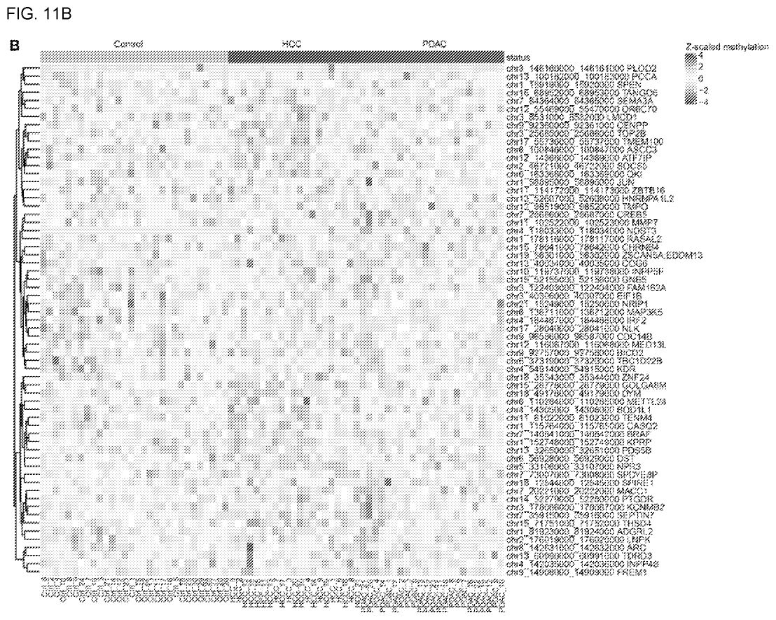

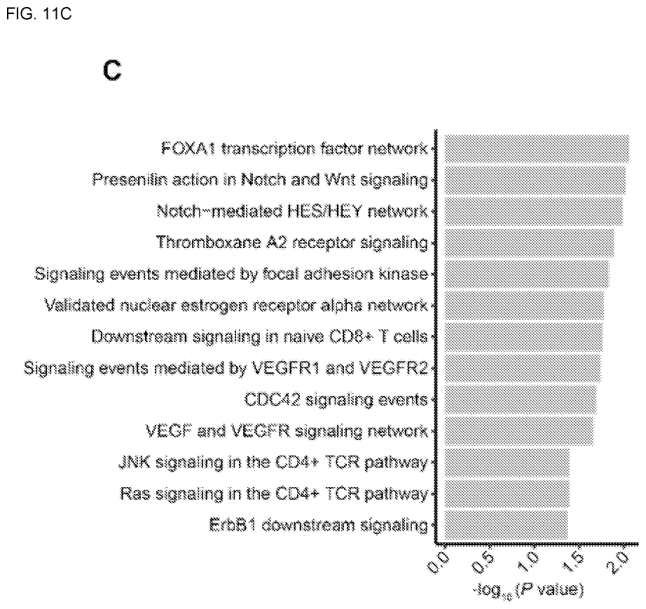

It was first confirmed that cfDNA fragmentation patterns detected with cfTAPS are concordant with cfDNA fragmentation pattern generated by whole-genome sequencing (WGS), with the dominant peak at 167 bp, a secondary peak at ˜320 bp and smaller peaks below 167 bp with 10 bp periodicity, reflecting nucleosomal fragmentation patterns (FIG. 3C; raw data pertaining to fragment length distribution in each individual are not included in the present disclosure but can be made available upon request). By contrast, fragmentation patterns were clearly different in previously published cfDNA WGBS, as the 10 bp oscillations in the cfDNA fragmentation profile were lost, presumably due to DNA damage (FIG. 10A). Consistent with previous cfDNA WGS, results indicated that cancer patients have a higher frequency of cfDNA fragments below 150 bp (Kruskal-Wallis test, HCC: P value 6.871e-06, PDAC: P value 0.006731) and a lower proportion of long fragments between 310-500 bp (Kruskal-Wallis test, HCC: P value 2.627e-07, PDAC: P value 1.263e-06) compared to non-cancer controls (FIG. 3D), further confirming the faithful preservation of cfDNA fragmentation information in cfTAPS.

A new approach was then developed for characterization of cfDNA fragmentation profiles using cfTAPS. Briefly, the cfDNA fragmentation distribution was divided into 10 bp bins and calculated the proportion of fragments in each 10 bp bin (FIG. 3C). It was found that cfDNA long fragments (300-500 bp) length proportion in 10 bp bins separated PDAC and HCC from controls in unsupervised analysis by PCA (FIG. 3E). Results further showed that this cfDNA fragmentation signature can be used to distinguish HCC and PDAC from non-cancer controls with high accuracy (HCC AUC=0.92, PDAC AUC=0.84) (FIGS. 10B, 10C, 10E, and 10F). However, this approach was less accurate at distinguishing cancer from cirrhosis and pancreatitis compared to methylation-based classifiers (FIGS. 10D and 10G), suggesting fragmentation information is less cancer-specific.

### Example 6

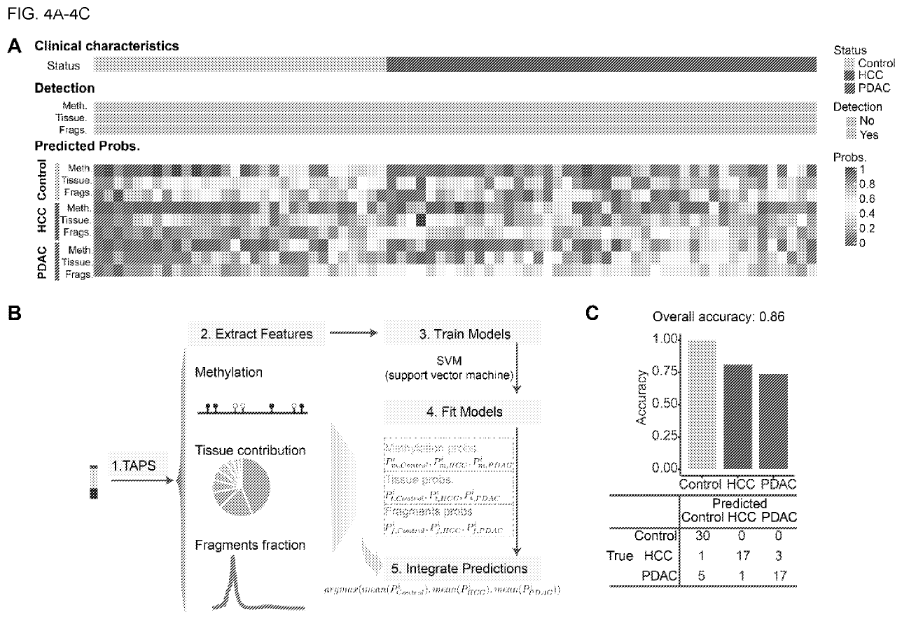

Multi-cancer detection with cfTAPS. Experiments were then conducted to investigate the utility of cfTAPS for multi-cancer detection. The top 5 DMRs of each pairwise comparison (non-cancer controls versus HCC, non-cancer controls versus PDAC, HCC versus PDAC) were selected as features in the multi-cancer differential methylation model. A Support Vector Machine (SVM) model was trained to estimate the respective probability that the blood sample came from each group. Similar models were built using tissue contribution and fragmentation profile. Using LOO cross validation, results indicated that the methylation model can achieve an overall accuracy of 0.77, which outperforms the tissue contribution model and fragmentation profile model (accuracy 0.62 and 0.46, respectively, FIG. 4A, FIG. 11A).

To further enhance the multi-cancer predictive model, a multimodal classifier was built that combined differential methylation, tissue contribution and fragment profile (FIG. 4B). This integrated model took the averaged scores across the three modalities and used the most confident prediction for each sample. The overall accuracy of the combined model was 0.86 (64 out of 74 were classified correctly) and the accuracy for distinguishing controls from any cancer type is 0.92 (FIG. 4C), which highlights the benefits of incorporating multimodal information for cancer type prediction. Finally, the DMRs used for multi-cancer prediction were explored (FIG. 111B; data pertaining to methylation features used for HCC, PDAC, and Control predictions are not included in the present disclosure but can be made available upon request). Interestingly, results indicated that the nearby genes of these regions were enriched in Notch and Wnt signalling, and EGFR (ErbB) signalling, which provides biological support for these potential multi-cancer biomarkers (FIG. 11C).

What is claimed is:

1. A method of obtaining a methylation signature, the method comprising:
isolating cell free DNA (cfDNA) from a sample;
preparing a sequencing library comprising the cfDNA; and
performing TET-assisted Pyridine Borane Sequencing (TAPS) on the sequencing library to obtain a whole-genome methylation signature of the cfDNA.

2. The method of claim 1, wherein the unique mapping rate resulting from TAPS is at least 80% and/or the unique deduplicated mapping rate is at least 70%.

3. The method of claim 1 or claim 2, wherein preparing the sequencing library comprises ligating sequencing adapters to the isolated cfDNA.

4. The method of any of claims 1 to 3, wherein carrier DNA is added to the sequencing library prior to performing TAPS.

5. The method of any of claims 1 to 4, wherein the method further comprises identifying at least one methylation biomarker from the cfDNA whole-genome methylation signature, and determining whether the methylation biomarker is indicative of cancer.

6. The method of claim 5, wherein the methylation biomarker comprises a differentially methylated region (DMR).

7. The method of claim 6, wherein the method further comprises classifying the sample based on the DMR as compared to a reference DMR.

8. The method of claim 7, wherein the reference DMR corresponds to a non-cancerous control, or a cancerous control.

9. The method of any of claims 1 to 4, wherein the method further comprises identifying at least one methylation biomarker from the cfDNA whole-genome methylation signature, and determining a tissue-of-origin corresponding to the methylation biomarker.

10. The method of claim 9, wherein the method further comprises classifying the sample based on the tissue-of-origin biomarker.

11. The method of any of claims 1 to 4, wherein the method further comprises identifying a DNA fragmentation profile and determining whether the fragmentation profile is indicative of cancer.

12. The method of any of claims 1 to 4, wherein the method further comprises identifying at least one sequence variant from the cfDNA, and determining whether the sequence variant is indicative of cancer.

13. The method of any of claims 1 to 12, wherein performing TAPS on the sequencing library to obtain the whole-genome methylation signature comprises identifying 5mC modifications in the cfDNA and providing a quantitative measure for frequency of the 5mC modifications.

14. The method of any of claims 1 to 12, wherein performing TAPS on the sequencing library to obtain the whole-genome methylation signature comprises identifying 5hmC modifications in the cfDNA and providing a quantitative measure for frequency of the 5hmC modifications.

15. The method of any of claims 1 to 12, wherein performing TAPS on the sequencing library to obtain the whole-genome methylation signature comprises identifying 5caC modifications in the cfDNA and providing a quantitative measure for frequency of the 5caC modifications.

16. The method of any of claims 1 to 12, wherein performing TAPS on the sequencing library to obtain the whole-genome methylation signature comprises identifying 5fC modifications in the cfDNA and providing a quantitative measure for frequency of the 5fC modifications.

17. A method of determining whether a subject has cancer using any of the methods of claims 1 to 16.

18. The method of claim 17, wherein the cancer comprises hepatocellular carcinoma (HCC) or pancreatic ductal adenocarcinoma (PDAC).

19. A method of determining whether a subject has early stage cancer using any of the methods of claims 1 to 16.

20. The method of claim 19, wherein the early stage cancer comprises early stage hepatocellular carcinoma (HCC) or early stage pancreatic ductal adenocarcinoma (PDAC).

21. A multimodal method of analyzing cfDNA in a patient sample comprising:
isolating cfDNA from a patient sample;
converting 5mC and/or 5hmC residues in the sample to DHU residues to provide a modified cfDNA sample;
sequencing the modified cfDNA sample to identify methylated regions in the sample, wherein a cytosine (C) to thymine (T) transition or a cytosine (C) to DHU transition in the modified cfDNA sample as compared to an unmodified reference cfDNA provides the location of either a 5mC or 5hmC in the cfDNA; and
performing one or more additional analytical steps on the modified cfDNA selected from the group consisting of:
a) determining copy number variation of one or more targets in the modified cfDNA sample;
b) determining the tissue of origin or one or more targets in the modified cfDNA sample;
c) determining the fragmentation profile of the modified cfDNA sample; and
d) identifying one or more single nucleotide mutations in the modified cfDNA sample.

22. The method of claim 21, wherein the step of sequencing the modified cfDNA sample to identify methylated regions in the sample comprising identifying at least one differentially methylated region (DMR).

23. The method of claim 22, wherein the method further comprises classifying the sample based on the DMR as compared to a reference DMR.

24. The method of claim 23, wherein the reference DMR corresponds to a non-cancerous control, or a cancerous control.

25. The method of claim 21, wherein the step of determining copy number variation (CNV) of one or more targets in the modified cfDNA sample comprises determining the observed read count for a target sequence across the genome by dividing the reference genome into bins and counting the number of reads in each bin.

26. The method of claim 25, wherein the presence of copy number aberrations of greater than 500 kb is indicative of CNV in a patient.

27. The method of claim 21, wherein the step of determining the tissue of origin or one or more targets in the modified cfDNA sample comprises tissue deconvolution of data obtained from sequencing the modified cfDNA sample.

28. The method of claim 27, wherein the tissue deconvolution comprises comparing DNA methylation value identified in the modified cfDNA sample with reference DMRs from two or more different tissues.

29. The method of claim 21, wherein the step of determining the fragmentation profile of the modified cfDNA sample comprises classifying the fragment length and periodicity of fragments in the modified cfDNA sample.

30. The method of claim 28, wherein classifying the length and periodicity of fragments in the modified cfDNA sample further comprises calculating the proportion of cfDNA fragments of from 300 to 500 bp in 10 bp length range bins.

31. The method of claim 21, wherein the step of identifying one or more single nucleotide mutations in the modified cfDNA sample further comprises distinguishing C to T SNPs from 5mC or 5hmC at a specific position in the cfDNA by comparing sequencing results after TAPS, wherein the presence of a T read at the specific position in a compliment to the original bottom strand of the cfDNA is indicative of a C to T SNP and the presence of a C read at the specific position in a compliment to the original bottom strand of the cfDNA is indicative of 5mC or 5hmC.

32. The method of any one of claims 21 to 31, wherein two or more of steps a, b, c and d are performed on the modified cfDNA.

33. The method of any one of claims 21 to 31, wherein three or more of steps a, b, c and d are performed on the modified cfDNA.

34. The method of any one of claims 21 to 31, wherein all of steps a, b, c and d are performed on the modified cfDNA.

35. The method of any one of claims 21 to 34, wherein the unique mapping rate resulting from the sequencing step is at least 80% and/or the unique deduplicated mapping rate is at least 70%.

36. The method of any one of claims 21 to 35, wherein the sequencing step further comprises preparing a sequencing library comprising the cfDNA by ligating sequencing adapters to the isolated cfDNA.

37. The method of any of claims 21 to 36, wherein carrier DNA is added to the cfDNA.

38. The method of any of claims 21 to 37, wherein the method provides a cfDNA whole-genome methylation signature and the method further comprises identifying at least one methylation biomarker from the cfDNA whole-genome methylation signature, and determining whether the methylation biomarker is indicative of cancer.

39. The method of any of claims 21 to 38, further comprising identifying 5mC modifications in the cfDNA and providing a quantitative measure for frequency of the 5mC modifications.

40. The method of any of claims 21 to 39, further comprising identifying 5hmC modifications in the cfDNA and providing a quantitative measure for frequency of the 5hmC modifications.

41. The method of any of claims 21 to 40, further comprising identifying 5caC modifications in the cfDNA and providing a quantitative measure for frequency of the 5caC modifications.

42. The method of any of claims 21 to 41, further comprising identifying 5fC modifications in the cfDNA and providing a quantitative measure for frequency of the 5fC modifications.

43. The method of any one claims 21 to 42, wherein the step of converting 5mC and/or 5hmC residues in the sample to DHU residues to provide a modified cfDNA sample comprises oxidizing 5mC and/or 5hmC residues to provide 5caC and/or 5fC residues and reducing the 5caC and/or 5fC residues to DHU residues.

44. The method of claim 43, wherein the step of oxidizing 5mC and/or 5hmC residues to provide 5caC and/or 5fC residues comprises treatment of the sample with a Tet enzyme.

45. The method of claim 43, wherein the step of oxidizing 5mC and/or 5hmC residues to provide 5caC and/or 5fC residues comprises treatment of the sample with a chemical oxidizing agent so that one or more 5fC residues are generated.

46. The method of any one of claims 43 to 45, wherein the step of reducing the 5caC and/or 5fC residues to DHU residues comprises treatment of the sample with a borane reducing agent.

47. A method of determining whether a subject has cancer using any of the methods of claims 21 to 46.


SEQUENCE LISTING (5 sequences)

SEQ ID NO: 1 - DNA, 2005 bp, unidentified
  source 1..2005: mol_type = genomic DNA
aggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatataaagttcctgagttcgattcgtccacaattaaaaatatctcttctgcaaaaggcctggacgtttgggacagctggccattacaaaacactgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgcctagcttcctgctgaacatcaaaggcaagaaaacatctgttgtcaaagacagcatccttgaacaaggacaattaacagttaacaaataaaaacgcaaaagaaaatgccgatatcctattggcattgacggtctccagtaaaggtggatacggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttgg

SEQ ID NO: 2 - DNA, 22 bp, synthetic construct
  source 1..22: mol_type = other DNA
aggcaactttatgcccatgcaa

SEQ ID NO: 3 - DNA, 24 bp, synthetic construct
  source 1..24: mol_type = other DNA
ccaaggggttatgctagttattgc

SEQ ID NO: 4 - DNA, 33 bp, synthetic construct
  source 1..33: mol_type = other DNA
acactctttccctacacgacgctcttccgatct

SEQ ID NO: 5 - DNA, 20 bp, synthetic construct
  source 1..20: mol_type = other DNA
gatcggaagagcacacgtct